(19) United States
(12) Patent Application Publication
(10) Pub. No.: US 2024/0209449 A1
(43) Pub. Date: Jun. 27, 2024

(54) METHODS AND SYSTEMS TO IDENTIFY A LUNG DISORDER

(71) Applicant: Veracyte, Inc., South San Francisco, CA (US)

(72) Inventors: Jing Huang, South San Francisco, CA (US); Lori Lofaro, South San Francisco, CA (US); P. Sean Walsh, South San Francisco, CA (US); Jie Ding, South San Francisco, CA (US); Jianghan Qu, South San Francisco, CA (US); Marla Johnson, South San Francisco, CA (US); Giulia C. Kennedy, San Francisco, CA (US)

(21) Appl. No.: 18/477,331

(22) Filed: Sep. 28, 2023

Related U.S. Application Data
(63) Continuation of application No. PCT/US2022/022192, filed on Mar. 28, 2022.
(60) Provisional application No. 63/167,598, filed on Mar. 29, 2021.

(51) Int. Cl. C12Q 1/6886; G16H 50/30

(52) U.S. Cl. CPC C12Q 1/6886; G16H 50/30; C12Q 2600/118; C12Q 2600/158

(57) ABSTRACT

Provided herein are methods and systems for analyzing a sample of a subject by using a trained algorithm to evaluate and classify the sample as indicating a risk of having or developing cancer.

### CROSS-REFERENCE

This application is a continuation application of International Patent Application No. PCT/US2022/022192, filed Mar. 28, 2022, which claims priority to U.S. Provisional Application 63/167,598, filed Mar. 29, 2021, each of which is entirely incorporated herein by reference.

### BACKGROUND

There are various types of lung conditions, such as diseases that may affect the lung or airways of subject. Examples of lung diseases include, but are not limited to lung cancer, COPD, cystic fibrosis, chronic bronchitis, asthma, pneumonia, idiopathic pulmonary fibrosis, and pulmonary edema.

Lung cancer is a type of cancer that may be due to abnormal tissue grown in a lung of a subject. Lung cancer may have a genetic basis (e.g., the subject is genetically predisposed to abnormal cell growth in the lungs of the subject), environmental basis (e.g., exposure to pollutants, such as cigarette smoke), or both. Lung cancer is the deadliest form of cancer in the United States and the world. An estimated 221,000 new lung cancer diagnoses are expected in the United States in 2015, and approximately 158,000 men and women are expected to fall victim to the disease during the same time period. The high mortality rate is due, in part, to a failure in 70% of patients to detect lung cancer when it is localized and surgical resection remains feasible. Additionally, diagnosis procedures for lung cancer are often painful and invasive.

A clinical gap remains in the assessment of indeterminate pulmonary nodules (PN) in individuals at increased risk of lung cancer due to smoking. Clinical guidelines exist for small incidental nodules (<8 mm), nodules identified in lung cancer screening, and larger PN (8-30 mm). The guidelines recommend an individualized approach to PN management starting with an estimate of the probability of malignancy using risk factors, radiographic features, and validated clinical risk model calculators. Management approaches in clinical practice are often inconsistent with published guidelines, and the utility of risk model calculators decreases when applied outside the inclusion criteria used to validate the models. A non-invasive tool to more accurately risk stratify patients could facilitate guideline adherence and more timely diagnosis of early-stage cancer, while reducing the need for unnecessary procedures in those with benign disease. A lung cancer molecular biomarker could serve as such a tool.

Methods currently available for detecting lung conditions, such as lung cancer, may not be able to (i) to assess a subject's risk for developing a lung condition or (ii) to detect many lung conditions in their early stages. Additionally, such methods may involve highly invasive and painful procedures.

### SUMMARY

For individuals who smoke or have previously smoked, use of genomic information may improve risk stratification accuracy beyond clinical factors. It is well established that genomic changes associated with lung cancer can be detected in benign respiratory epithelial cells. A genomic classifier utilizing brushings obtained from cytologically benign bronchial epithelial cells has been shown to accurately predict ROM in patients with a suspicious lung lesion and a non-diagnostic bronchoscopy. This “field of injury” principal is shown to be detectable in nasal epithelial cells. Disclosed herein is a nasal clinical-genomic classifier developed using RNA whole-transcriptome sequencing and machine learning which can serve as a non-invasive tool for lung cancer risk assessment in individuals who smoke or have previously smoked with a pulmonary nodule (PN).

Disclosed herein is a method for determining that a subject is not at risk of having lung cancer, comprising (a) assaying a biological sample from a nasal passageway of said subject for a level of expression, and (b) processing said level of expression to determine that said subject is not at risk of having said lung cancer at a specificity of at least 51%. Step (b) can be performed at a sensitivity of at least 95%. The biological sample can be a sample of airway epithelial cells. The airway epithelial cells can be obtained by nasal swab. The lung cancer can comprise one or more of non-small cell lung cancer, a small cell lung cancer, a lung carcinoid tumor, or a bronchial carcinoid tumor. The non-small cell lung cancer can comprise one or more of an adenocarcinoma, a squamous cell carcinoma, or a large cell carcinoma. Processing can comprise correlating one or more additional levels of expression with one or more genomic index. The one or more genomic index can comprise a blood contamination index. The blood contamination index can comprise an expression level of hemoglobin subunit beta. The one or more genomic index can comprise a smoking duration index. The smoking duration index can comprise an expression level of one or more genes selected from Table 1. The smoking duration index can comprise an expression level of one or more genes selected from the group consisting of: AC074091.1, ACTL10, ADRA2B, AGT, ALDOC, AMACR, AOX1, APEH, APOPT1, ARHGEF35, ARNTL, ATF7IP2, ATP2A3, BBOX1, BHLHE40-AS1, BNIP3, BOLA1, BPI, C11orf68, C12orf65, C1QL2, C21orf128, C2orf73, CACNA1B, CAPG, CAPN9, CDC25A, CDC42P6, CDCA2, CDCP1, CDHR1, CDHR2, CDK5, CDNF, CMTM2, COG1, COL1A1, COL5A3, CORO2B, CST7, CTD-2555O16.2, CTD-2555O16.4, CTGLF12P, CTNS, CTSF, CXCL12, CYP7B1, DBI, DDO, DDT, DLL1, DOCK3, DRD4, EDIL3, EFHB, ETFDH, EVA1A, FAM184A, FAM189B, FLT1, FOXC2, FTCDNL1, GALNT16, GET4, GLB1L3, GNAL, GNG4, GOLGA8O, GOT1, HARBI1, HAUS4, HCAR3, HERC2P2, HIST1H3E, HIST1H4F, HLA-J, HORMAD1, HSF4, HSF5, IGF2BP2, ISYNA1, KCNMB3, KCNQ3, KCTD10, KDR, KIAA0513, KRT39, KRT40, KRTAP5-7, LOXHD1, LTBP1, LUZP2, LYRM5, MAD2L1BP, MMD, MMP1, MPP7, MRM1, MRPS6, MRVI1-AS1, MUC6, MUT, MVB12A, NAMPTL, NBR2, NDUFA6, NDUFAF6, NDUFS7, NEFH, NLRP2, NME6, NPSR1, NUDT7, OLFM1, ORAOV1, PALM3, PAPSS1, PCDHA12, PCDHA13, PCDHB11, PCDHB16, PDPR, PEX11A, PIAS2, PIPOX, PLAG1, PLG, PMP22, PMS2P5, POLR2M, PPFIA3, PPP1R42, PRPF38B, PTGER4, RANGRF, RBMS3, RIMBP2, RIMKLA, RND2, RP11-163E9.2, RP11-171I2.2, RP11-171I2.4, RP11-345J4.8, RP11-461A8.1, RP11-477D19.2, RP11-522I20.3, RP11-695J4.2, RPL9, RUSC1, SCN11A, SDHAF2, SEMA3F, SEPT7P9, SFRP2, SH3GL3, SLAMF6, SLC22A3, SLC37A2, SLC48A1, SLC6A13, SNORD101, SP6, SPINK1, STAG3L2, STXBP5L, TEKT4, TERF2, TF, TFAP2C, TMEM200C, TMEM213, TMTC4, TP53I11, TTC39B, TTLL13, TWF2, TYRO3, UBAP1L, WDR53, WIPF3, ZFP2, ZFP28, ZNF232, ZNF576, and ZNF624. The one or more genomic index can comprise a smoking status index. The smoking status index can comprise an expression level of one or more genes selected from Table 1. The smoking status index can comprise an expression level of one or more genes selected from the group consisting of: ACVRL1, AHRR, AP1S3, ARRDC4, B3GNT6, BAALC, BPIFB2, CACNA2D3, CCDC69, CCDC88A, CD163L1, CDK5RAP2, CIT, CLIC5, CMTM7, CNGB1, COL1A2, COL3A1, COL6A3, CPE, CPNE8, CRNN, CYP2A13, CYP4X1, EDC3, ENC1, ENTPD8, FHL1, FOXE1, GAD1, GLDN, GLYATL2, GRAMD2, GSTO2, hsa-mir-7162, HSF4, ICA1, IGF1, IL36A, JAKMIP3, KPRP, LCE3D, LRRC31, MAMDC2, MGP, MMP7, MPST, NOL3, NOX4, NRIP1, OCA2, PANX2, PBX3, PRKAR2B, RAMP1, RDH10, RHCG, RNF175, RPTN, SAA1, SAA2, SAMHD1, SERPINE2, SETD7, SLC16A12, SLC28A2, SLPI, TGM3, TGM6, TIPARP, TMEM45B, TRHDE, TRNAU1AP, UCHL1, USH1C, USP54, WNT5A, and ZKSCAN1. The one or more genomic index can comprise a cell type normalization index. The processing can comprise regressing out said one or more additional levels of expression associated with said cell type normalization index. The one or more genomic index can comprise a genomic gender index. The genomic gender index can comprise one or more of USP9Y, RPS4Y1, UTY, DDX3Y, or KDM5D. The method can further comprise measuring one or more additional levels of expression to determine an integrity of ribonucleic acid (RNA) in said sample. The method can further comprise measuring one or more clinical covariates comprising one or more of age, nodule length, nodule spiculation, or pack years. Pack years can be identified as less than 20 years, between 20 years sand 50 years, or greater than 50 years. Processing can comprise applying a trained classifier. The trained classifier can be trained using gene expression data from subjects diagnosed with lung cancer. The subjects diagnosed with lung cancer can include subjects with lung nodule sizes between 6 mm and 30 mm in diameter. The subjects diagnosed with lung cancer can include subjects with lung nodule sizes less than 6 mm in diameter. The subjects diagnosed with cancer can include subjects with unknown lung nodule sizes.

Disclosed herein is a method for determining a likelihood that a subject is free of a cancer, comprising (a) assaying a sample of said subject for a cancer marker and (b) processing said cancer marker to determine that said subject is free of said cancer at a likelihood of at least 85%. The likelihood can be determined with a specificity of at least 51%. The likelihood can be determined with a selectivity of at least 95%. The likelihood can be determined with a negative predictive value of greater than 90%. The sample can comprise airway epithelial cells. The airway epithelial cells can be obtained by nasal swab. The cancer can be lung cancer. The lung cancer can comprise one or more of non-small cell lung cancer, a small cell lung cancer, a lung carcinoid tumor, or a bronchial carcinoid tumor. The non-small cell lung cancer can comprise one or more of adenocarcinoma, squamous cell carcinoma, or large cell carcinoma. Processing can comprise correlating one or more additional markers with one or more genomic index. The one or more genomic index can comprise a blood contamination index. The one or more genomic index can comprise a smoking duration index. The one or more genomic index can comprise a smoking status index. The one or more genomic index can comprise a cell type normalization index. Processing can comprise regressing out said one or more additional marker levels associated with said cell type normalization index. The one or more genomic index can comprise a genomic gender index. The genomic gender index can comprise one or more of USP9Y, RPS4Y1, UTY, DDX3Y, or KDM5D. The one or more additional markers can be ribonucleic acid (RNA). The method can further comprise measuring one or more additional markers to determine an integrity of said cancer marker in said sample. The cancer marker can be ribonucleic acid (RNA). RNA can comprise mRNA, microRNA (miRNA), sRNA, siRNA, transfer RNA, and ribosomal RNA,

The method can further comprise measuring one or more clinical covariates comprising one or more of age, nodule length, nodule spiculation, or pack years. Pack years can be identified as less than 20 years, between 20 years sand 50 years, or greater than 50 years. Processing can comprise applying a trained classifier. The trained classifier can be trained using gene expression data from subjects diagnosed with cancer. The subjects diagnosed with cancer can include subjects with lung nodule sizes between 6 mm and 30 mm in diameter. The subjects diagnosed with cancer can include subjects with lung nodule sizes greater than 30 mm in diameter. The subjects diagnosed with cancer can include subjects with lung nodule sizes less than 6 mm in diameter. The subjects diagnosed with cancer can include subjects with unknown lung nodule sizes.

Disclosed herein is a system for screening a subject for a lung condition, comprising: one or more computer databases comprising health or physiological data of a subject; and one or more computer processors that are individually or collectively programmed to (i) assay a biological sample from a nasal passageway of said subject for a level of expression, and (ii) process said level of expression to determine that said subject is not at risk of having said lung condition at a specificity of at least 51%.

Disclosed herein is a system for screening a subject for a lung condition comprising: one or more computer databases comprising health or physiological data of a subject; and one or more computer processors that are individually or collectively programmed to (i) assay a biological sample from a nasal passageway of said subject for a level of expression, and (ii) process said level of expression to determine that said subject is free of said lung condition at a likelihood of at least 85%.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a graph of the candidate classifier score separation between nasal swab samples associated with benign nodules and nasal swab samples associated with malignant samples as compared to pure blood samples and brushing samples contaminated with blood.

FIG. 2 shows a graph of the index score separation between nasal swab samples and bronchial brushing samples within each database compared to bronchial brushing samples mixed with increasing amounts of blood.

FIG. 3 shows a plot of the number of unique cDNA fragments associated with cell type PC1 versus an estimated library size for cohorts in the cohort A and cohort B databases, and whether those cohorts are associated with nodules that are benign or malignant for lung cancer.

FIG. 4 shows a plot of median cross-validation (CV) scores of samples analyzed by a classifier versus a concentration of RNA in the sample.

FIG. 5A-C show plots of the effect of gene expression regression on training sample scores.

FIG. 6 shows a plot of the score normalization achieved in expression data from the COHORT A and Cohort B database using cell type PC1.

FIG. 7A is a plot of the variance of genes in cell types 1-10. FIG. 7B is a plot of the relative weights of ciliated genes and immune genes in cell type PC1 versus cell type PC2 in a gene expression profile.

FIG. 8A is a plot of the distribution of genes in cell type PC1 and PC2 by, demonstrating the spread of highly variable genes in each cell type. FIG. 8B is a series of plots showing the relative weights of only the genes identified as having a high variability, by cell type.

FIGS. 9A and 9B are plots showing the effect on weights applied to expression of a single genes across a plurality of training samples when the weights are calculated with and without genes that aren't associated with whether a sample is associated with a benign or malignant nodule, by regressing out the genes that aren't associated with whether a sample is associated with a benign or malignant nodule.

FIG. 10 shows a computer system as described herein.

FIG. 11 shows a comparison of the receiver operating characteristic (ROC) curves for the genomic smoking status index as applied to gene expression data normalized using the rb1 gene set and the rb1rc12 gene set.

FIG. 12 shows a comparison of the receiver operating characteristic (ROC) curves for the smoking duration index and the clinical smoking years covariate as applied to gene expression data without normalization, normalized using the rb1 gene set, and using the rb1rc12 gene set.

FIG. 13 shows the scoring associated with biological gender using the genomic gender index on data without normalization and data normalized using the rb1 gene set and the rb1rc12 gene set.

FIG. 14 shows a graph of TPR (true positive rate) versus FPR (false positive rate) for gene expression data normalized using the rb1 gene set and the rb1rc12 gene set.

FIG. 15 shows a flow chart of the two-layer classifier model and a visual representation of which samples from each database are captured in each layer.

FIG. 16 shows a receiver operating characteristic (ROC) curve for the Model A classifier.

FIG. 17 shows the scoring by Model A of samples associated with benign or malignant nodules in each database and overall after each layer of the model.

FIG. 18 shows a receiver operating characteristic (ROC) curve for the Model B classifier.

FIG. 19 shows the scoring by Model B of samples associated with benign or malignant nodules in each database and overall after each layer of the model.

FIG. 20 shows a receiver operating characteristic (ROC) curve for the Model C classifier.

FIG. 21 shows the scoring by Model C of samples associated with benign or malignant nodules in each database and overall after each layer of the model.

FIG. 22 shows a receiver operating characteristic (ROC) curve for the Model D classifier.

FIG. 23 shows the scoring by Model D of samples associated with benign or malignant nodules in each database and overall after each layer of the model.

FIG. 24 shows a receiver operating characteristic (ROC) curve for the Model E classifier.

FIG. 25 shows the scoring by Model E of samples associated with benign or malignant nodules in each database and overall.

FIG. 26 shows a receiver operating characteristic (ROC) curve for the Model F classifier.

FIG. 27 shows the scoring by Model F of samples associated with benign or malignant nodules in each database and overall.

FIG. 28 shows a graph of the number of samples associated with a patient identified as having a nodule of a particular length wherein dark grey bars are samples from the Cohort A database and light grey bars and samples from the Cohort B Database.

FIG. 29 shows a consort diagram of training and validation sets.

FIG. 30 shows alluvial plots showing distribution of benign and malignant nodules into high, intermediate, and low-risk categories for A. the primary validation set, B. the primary validation set and secondary prior cancer set combined, C. the primary validation set extrapolated to a cancer prevalence of 25%, and D. the primary validation set and prior cancer set combined extrapolated to a cancer prevalence of 25%.

FIG. 31 shows a consort diagram of the prior cancer set.

FIG. 32 shows a Sankey plot showing distribution of the classification results of the nasal classifier validation cohort and their corresponding classifier result in a population extrapolated to 25% cancer prevalence of malignancy.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. A human may be an infant, a toddler, a child, a young adult, an adult or a geriatric. The human can be at least about 1, 2, 5, 10, 20, 30, 40, 50, 60, 65, 70, 75, 80 years or more of age. The human may be suspected of having a disease, such as, e.g., lung cancer. Alternatively, the human may be asymptomatic.

The subject may have or be suspected of having a disease, such as cancer. The subject may be a smoker, a former smoker or a non-smoker. The subject may have a personal or family history of cancer. The subject may have a cancer-free personal or family history. The subject may be a patient, such as a patient being treated for a disease, such as a cancer patient. The subject may be predisposed to a risk of developing a disease such as cancer. The subject may be in remission from a disease, such as a cancer patient. The subject may be healthy. The subject may exhibit one or more symptoms of lung cancer or other lung disorder (e.g., emphysema, COPD). For example, the subject may have a new or persistent cough, worsening of an existing chronic cough, blood in the sputum, persistent bronchitis or repeated respiratory infections, chest pain, unexplained weight loss and/or fatigue, or breathing difficulties such as shortness of breath or wheezing. The subject may have a lesion, which may be observable by computer-aided tomography (“CT”) or chest X-ray. The subject may have a suspicious lesion or nodule, which may be observable by low-dose computer-aided tomography (“LD-CT”). The suspicious lesion or nodule may be identified in a lobe of a lung of the subject. The subject may be an individual who has undergone a bronchoscopy or who has been identified as a candidate for bronchoscopy (e.g., because of the presence of a detectable lesion, or suspicious or inconclusive imaging result). The subject may be an individual who has undergone an indeterminate or non-diagnostic bronchoscopy. The subject may be an individual who has undergone an indeterminate or non-diagnostic bronchoscopy and who has been recommended to proceed with an invasive lung procedure (e.g., transthoracic needle aspiration, mediastinoscopy, lobectomy, or thoracotomy) based upon the indeterminate or nondiagnostic bronchoscopy. The terms, “patient” and “subject” are used interchangeably herein. The subject may be at risk for developing lung cancer. The subject may be at risk for suffering from a recurrence of lung cancer. The subject may have lung cancer and the assays and methods disclosed herein may be used to monitor the progression of the subject's disease or to monitor the efficacy of one or more treatment regimens.

The subject can be suspected of having a lung disorder. The lung disorder can be an interstitial lung disease (ILD). “Interstitial lung disease” or “ILD” (also known as diffuse parenchymal lung disease (DPLD)) as used herein refers to a group of lung diseases affecting the interstitium (the tissue and space around the air sacs of the lungs). ILD can be classified according to a suspected or known cause, or can be idiopathic. For example, ILD can be classified as caused by inhaled substances (inorganic or organic), drug induced (e.g., antibiotics, chemotherapeutic drugs, antiarrhythmic agents, statins), associated with connective tissue disease (e.g., systemic sclerosis, polymyositis, dermatomyositis, systemic lupus erythematous, rheumatoid arthritis), associated with pulmonary infection (e.g., atypical pneumonia, Pneumocystis pneumonia (PCP), tuberculosis, Chlamydia trachomatis, Respiratory Syncytial Virus), associated with a malignancy (e.g., Lymphangitic carcinomatosis), or can be idiopathic (e.g., sarcoidosis, idiopathic pulmonary fibrosis, Hamman-Rich syndrome, antisynthetase syndrome). “ILD Inflammation” as used herein refers to an analytical grouping of inflammatory ILD subtypes characterized by underlying inflammation. These subtypes can be used collectively as a comparator against IPF and/or any other non-inflammation lung disease subtype. “ILD inflammation” can include HP, NSIP, sarcoidosis, and/or organizing pneumonia. “Idiopathic interstitial pneumonia” or “IIP” (also referred to as noninfectious pneumonia” refers to a class of ILDs which includes, for example, desquamative interstitial pneumonia, nonspecific interstitial pneumonia, lymphoid interstitial pneumonia , cryptogenic organizing pneumonia, and idiopathic pulmonary fibrosis. “Idiopathic pulmonary fibrosis” or “IPF” as used herein refers to a chronic, progressive form of lung disease characterized by fibrosis of the supporting framework (interstitium) of the lungs. By definition, the term is used when the cause of the pulmonary fibrosis is unknown (“idiopathic”). Microscopically, lung tissue from patients having IPF shows a characteristic set of histologic/pathologic features known as usual interstitial pneumonia (UIP), which is a pathologic counterpart of IPF. “Nonspecific interstitial pneumonia” or “NSIP” is a form of idiopathic interstitial pneumonia generally characterized by a cellular pattern defined by chronic inflammatory cells with collagen deposition that is consistent or patchy, and a fibrosing pattern defined by a diffuse patchy fibrosis. In contrast to UIP, there is no honeycomb appearance nor fibroblast foci that characterize usual interstitial pneumonia. “Hypersensitivity pneumonitis” or “HP” refers to also called extrinsic allergic alveolitis, (EAA) refers to an inflammation of the alveoli within the lung caused by an exaggerated immune response and hypersensitivity to as a result of an inhaled antigen (e.g., organic dust). “Pulmonary sarcoidosis” or “PS” refers to a syndrome involving abnormal collections of chronic inflammatory cells (granulomas) that can form as nodules. The inflammatory process for HP generally involves the alveoli, small bronchi, and small blood vessels. In acute and subacute cases of HP, physical examination usually reveals dry rales.

The term “disease,” as used herein, generally refers to any abnormal or pathologic condition that affects a subject. Examples of a disease include cancer, such as, for example, lung cancer. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein.

The term “disease diagnostic,” as used herein, generally refers to diagnosing or screening for a disease, to stratify a risk of occurrence of a disease, to monitor progression or remission of a disease, to formulate a treatment regime for the disease, or any combination thereof. A disease diagnostic can include a) obtaining information from one or more tissue samples from a subject, b) making a determination about whether the subject has a particular disease based on the information or tissue sample obtained, c) stratifying the risk of occurrence of the disease, or risk of malignancy, in the subject, including up- or down-classifying a risk of occurrence or malignancy for a subject (e.g., intermediate risk down-classified to low-risk, or intermediate risk up-classified to high risk), and, optionally, d) confirming whether the tissue sample from the subject is positive or negative for a lung disorder (e.g., lung cancer). The disease diagnostic may inform a particular treatment or therapeutic intervention for the disease. The disease diagnostic may also provide a score indicating for example, the severity or grade of a disease such as cancer, or the likelihood of an accurate diagnosis, such as via a p-value, a corrected p-value, or a statistical confidence indicator. The methods disclosed herein may also indicate a particular type of a disease.

The term “respiratory tract,” as used herein, generally refers to tissue found along the nose, mouth, throat, trachea, airway, bronchi, and/or lungs of a subject.

The term “homology,” as used herein, generally refers to calculations of homology or percent homology between two or more nucleotide or amino acid sequences that may be determined by aligning the sequences for comparison purposes (e.g., gaps can be introduced in the sequence of a first sequence). Nucleotides at corresponding positions may then be compared, and the percent identity between the two sequences may be a function of the number of identical positions shared by the sequences (i.e., % homology=# of identical positions/total # of positions×100). For example, if a position in the first sequence is occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. The length of a sequence aligned for comparison purposes may be at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 95%, of the length of the reference sequence.

The term “lung cancer,” as used herein, generally refers to a cancer or tumor of a lung or lung-associated tissue. For example, lung cancer may comprise a non-small cell lung cancer, a small cell lung cancer, a lung carcinoid tumor, or any combination thereof. A non-small cell lung cancer may comprise an adenocarcinoma, a squamous cell carcinoma, a large cell carcinoma, or any combination thereof. A lung carcinoid tumor may comprise a bronchial carcinoid. A lung cancer may comprise a cancer of a lung tissue such as a bronchiole, an epithelial cell, a smooth muscle cell, an alveoli, or any combination thereof. A lung cancer may comprise a cancer of a trachea, a bronchius, a bronchiole, a terminal bronchiole, or any combination thereof. A lung cancer may comprise a cancer of a basal cell, a goblet cell, a ciliated cell, a neuroendocrine cell, a fibroblast cell, a macrophage cell, a Clara cell, or any combination thereof.

The term “fragment,” as used herein, generally refers to a portion of a sequence, such as a subset that may be shorter than a full length sequence. A fragment may be a portion of a gene.

The term “amplification”, as used herein, generally refers to any process of producing at least one copy of a nucleic acid molecule. The terms “amplicons” and “amplified nucleic acid molecule” refer to a copy of a nucleic acid molecule and can be used interchangeably.

The term “machine learning algorithm” as used herein, generally refers to a computationally-based methodology, including an algorithm(s) and/or statistical model(s), that may perform a specific task without using explicit instructions, such as, for example, relying on patterns and inference. A machine learning algorithm may be an algorithm that has been trained or may be trained on at least one training set, which may be used to characterize a biomolecule profile. A machine learning algorithm may be a classifier of a disease or tissue type. A biomolecule profile may be a gene expression profile (e.g., a profile or mRNA or cDNA molecules derived from mRNA). A biomolecule profile may be a nucleic acid sequence profile, e.g., a profile of amino acid sequences, a profile of RNA and DNA sequences, a profile of DNA sequences, a profile of RNA sequences, or any combination thereof. The signals corresponding to certain expression levels, which may be obtained by, e.g., microarray-based hybridization or sequencing assays, may be t subjected to the classifier algorithm to classify the expression profile. Machine learning may be supervised or unsupervised. Supervised learning generally involves “training” a classifier to recognize the distinctions among classes and then “testing” the accuracy of the classifier on an independent test set. For new, unknown samples the classifier can be used to predict the class in which the samples belong.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Disclosed herein are non-invasive or minimally invasive assays and related methods that are useful for determining the pathological status of a sample obtained from a subject, which can be used for, as non-limiting examples, diagnosing lung disorder, such as lung cancer, or determining a subject's previous smoking status. Described herein are classifiers, assays and methods that can comprise determining the expression of one or more genes in sample obtained from a subject, for example, a nasal epithelial sample or a bronchial sample. In certain aspects the methods disclosed herein can comprise comparing the expression of one or more of the genes in a sample obtained from a subject to expression of the same genes in a sample of the same tissue type obtained from a control subject. In certain aspects, the assays described herein involves obtaining a sample from a subject's nasal epithelial cells. For example, cells may be taken from the airway of an individual that has been exposed to an airway pollutant (the “field of injury”). The airway pollutant can be cigarette smoke, smog, asbestos, inhaled medications, aerosols, etc. The airway may include a nasal passageway. In certain aspects, disclosed herein are methods of up- or down-classifying a risk of malignancy for lung cancer in a subject based on analyzing clinical or genomic features of the subject or a sample obtained from the subject. The sample may be obtained from a nasal passage and classification of such a sample may be used to identify a subject's risk of malignancy for lung cancer, allowing for assessment of risk for lung cancer without requiring invasive sampling procedures. In certain aspects, any of the methods disclosed herein further comprise identifying a blood contamination of a sample. In certain aspects, any of the methods disclosed herein further comprise identifying a ribonucleic acid integrity of a sample.

A sample may be provided or obtained from a subject. The sample can be obtained from a tissue separate from the tissue identified as having a suspicious lesion or nodule. For example, a suspicious lesion or nodule may be seen on a left lobe of a lung and the sample may be obtained from a right bronchus, an esophagus, a larynx, an oral tissue, or a nasal tissue of the subject. For example, a suspicious lesion or nodule may be seen on a right lobe of a lung and the sample may be obtained from a left bronchus, an esophagus, a larynx, an oral tissue, or a nasal tissue of the subject. For example, a suspicious lesion or nodule may be seen on a left bronchus and the sample may be obtained from a right bronchus, an esophagus, a larynx, an oral tissue, or a nasal tissue of the subject. For example, a suspicious lesion or nodule may be seen on a right bronchus and the sample may be obtained from a left bronchus, an esophagus, a larynx, an oral tissue, or a nasal tissue of the subject. The sample may comprise cells obtained from a portion of an airway, such as epithelial cells obtained from a portion of an airway. The sample may be a tissue sample removed from the subject, such as a tissue brushing, a swabbing, a tissue biopsy, an excised tissue, a fine needle aspirate, a tissue washing, a cytology specimen, a bronchoscopy, or any combination thereof. The sample may be provided or obtained from a subject who is using one or more inhaled medications. The inhaled medications may include, for example, bronchodilators, steroids, or a combination thereof.

The sample may be obtained from a subject who has been diagnosed with a lung disease. The subject may be diagnosed with an interstitial lung disease, idiopathic pulmonary fibrosis, usual interstitial pneumonia, non-usual interstitial pneumonia, non-specific interstitial pneumonia (NSIP), idiopathic interstitial pneumonia, hypersensitivity pneumonitis (HP), pulmonary sarcoidosis (PS), or COPD. The sample may be obtained from a subject identified at being at risk for a lung disorder based on one or more risk factors. In some embodiments, the one or more risk factors comprise: smoking; exposure to environmental smoke; exposure to radon; exposure to air pollution; exposure to radiation; exposure to an industrial substance; exposure to inhaled medications; inherited or environmentally-acquired gene mutations; a subject's age; a subject having a secondary health condition; or any combination thereof. In some embodiments, the subject has two or more risk factors. The subject may be identified as being in remission for a cancer. The cancer can be lung cancer. The sample can be obtained from a subject with a suspicious lesion or nodule identified by imaging analysis or physical examination. Imaging analysis can comprise MRI, CT-scan, low-dose CT scan, or X-ray.

The sample may be obtained or provided after a clinical sample is extracted from the subject. The clinical sample may be a sample that is obtained by biopsy, fine needle aspirate, cytology specimen, bronchial brushing, tissue washing, excised tissue, swabbing, or any combination thereof.

The sample may comprise cells obtained from a respiratory tract of the subject. The sample may be a nasal tissue, a bronchial tissue, a lung tissue, an esophageal tissue, a larynx tissue, an oral tissue or any combination thereof. The sample may comprise cells obtained from a nasal tissue, a bronchial tissue, a lung tissue, an esophageal tissue, a larynx tissue, an oral tissue or any combination thereof. The sample may be suspected or confirmed of evidencing a disease or disorder, such as a cancer or a tumor. For instance, an airway brushing sample (e.g., a bronchial brushing sample) may be obtained from a subject after results from a bronchoscopy are found to be inconclusive. In collecting an airway brushing sample, multiple brushing samples may be collected from a given field in the subject's airway.

Samples that are known or confirmed as evidencing a disease or disorder may be used for machine learning algorithm training purposes.

The sample obtained may have a variety of pathologies. The sample may be cytologically indeterminate. The sample may be cytologically normal. The sample may be an ambiguous or suspicious sample, such as a sample obtained by fine needle aspiration, a bronchoscopy, or other small volume sample collection method. The sample may be derived from an intact region of a patient's body receiving cancer therapy, such as radiation. The sample may be a tumor in a patient's body. The sample may comprise cancerous cells, tumor cells, malignant cells, non-cancerous cells (e.g., normal or benign cells), or a combination thereof. The sample may comprise invasive cells, non-invasive cells, or a combination thereof.

The sample may be a nasal tissue, a tracheal tissue, a lung tissue, a pharynx tissue, a larynx tissue, a bronchus tissue, a pleura tissue, an alveoli tissue, or any combination or derivative thereof. The sample may be a plurality of cells (e.g., epithelial cells) obtained by bronchial brushing. The sample may be a plurality of cells (e.g., lung tissue) obtained by biopsy. The sample may be a secretion comprising a plurality of cells (e.g., epithelial cells) obtained by swab or irrigation of a mucus membrane.

Samples may include samples obtained from: a subject having a pre-existing benign lung disease; a subject having chronic pulmonary infections; a subject having a suppressed immune system; a subject having an increased hereditary risk of developing a lung condition; a non-smoker having environmental exposure; or any combination thereof. Samples may be obtained from a plurality of different countries.

The sample may be an isolated and purified sample. The sample may be a freshly isolated sample. Cells from the freshly isolated sample may be isolated and cultured. The sample may comprise one or more cells. An isolated sample may comprise a heterogeneous mixture of cells. A sample may be purified to comprise a homogeneous mixture of cells. The sample may comprise at least about 100 cells, 1,000 cells, 5,000 cells, 10,000 cells, 20,000 cells, 30,000 cells, 40,000 cells, 50,000 cells, 60,000 cells, 70,000 cells, 80,000 cells, 90,000 cells, 100,000 cells, 150,000 cells, 200,000 cells, 250,000 cells, 300,000 cells, 350,000 cells, 400,000 cells, 450,000 cells, 500,000 cells, 550,000 cells, 600,000 cells, 650,000 cells, 700,000 cells, 750,000 cells, 800,000 cells, 850,000 cells, 900,000 cells, 950,000 cells, or more. The sample may comprise from about 30,000 cells to about 1,000,000 cells. The sample may comprise from about 20,000 cells to about 50,000 cells. The sample may comprise from about 100,000 cells to about 400,000 cells. The sample may comprise from about 400,000 cells to about 800,000 cells.

The sample may be collected from the same subject more than one time. Periodic sample collection may be performed to monitor a subject that is identified as being at risk for lung cancer or lung disease. For example, a first sample may be collected from a subject and a second sample may be collected about 1 year after the first sample has been collected. Samples may be collected from the same subject about: bi-weekly, weekly, bi-monthly, monthly, bi-yearly, yearly, every two years, every three years, every four years, or every five years. Samples may be collected annually from a subject. Results from the second sample may be compared to results of a first sample to monitoring a disease progression in the subject, an efficacy of a prescribed treatment or therapy, or a change in a risk of developing a condition, or any combination thereof.

### Gene Expression Analysis

Nucleic acid molecules may be amplified. The amplification reactions may comprise PCR-based methods, non-PCR based methods, or a combination thereof. Examples of non-PCR based methods may include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. PCR-based methods may include, but are not limited to, PCR, HD-PCR, Next Gen PCR, digital RTA, or any combination thereof. Additional PCR methods may include, but are not limited to, linear amplification, allele-specific PCR, Alu PCR, assembly PCR, asymmetric PCR, droplet PCR, emulsion PCR, helicase dependent amplification HDA, hot start PCR, inverse PCR, linear-after-the-exponential (LATE)-PCR, long PCR, multiplex PCR, nested PCR, hemi-nested PCR, quantitative PCR, real time PCR (RT-PCR) or quantitative PCR (qPCR), single cell PCR, and touchdown PCR.

RNA sequencing (such as exome enriched RNA sequencing or the sequencing of cDNA obtained from RNA) may generate short sequence fragments. RNA can be sequenced by first undergoing reverse transcription into cDNA (i.e. RT-qPCR, RT-PCR, qPCR). Following reverse transcription, the cDNA can be sequenced. Each fragment, or “read”, of a cDNA molecule can be used to measure levels of gene expression. RNA can comprise mRNA, microRNA (miRNA), sRNA, siRNA, transfer RNA, or ribosomal RNA,

Sequence identification methods may include sequence hybridization methods such as NanoString. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Nova Seq (Illumina), Digital Gene Expression (Helicos), Single Molecule Sequencing by Synthesis (SMSS)(Helicos), massively-parallel sequencing, Clonal Single Molecule Array (Solexa), shotgun sequencing, Maxim-Gilbert sequencing, primer walking, sequencing using PacBio, SOLiD, Ion Torrent, or Nanopore platforms and any other sequencing methods.

Sequencing may include sequencing technologies having increased throughput as compared to traditional Sanger- and capillary electrophoresis-based approaches, for example with the ability to generate hundreds of thousands of relatively small sequence reads at a time. Some examples of sequencing techniques include, but are not limited to, sequencing by synthesis, sequencing by ligation, and sequencing by hybridization.

Additional techniques may be used to detect various biomarkers in addition to gene fusions (e.g., DNA, cDNA, transcripts thereof, and related peptide sequences).

Epigenetic biomarkers (such as DNA methylation, such as 5-hydroxymethylated cytosine, 5-methylated cytosine, 5-carboxymethylated cytosine, or 5-formylated cytosine) may be detected by sequencing, microarrays, PCR, RT-PCR, qPCR, mass spectrometry (MS), Chromatin Immunoprecipitation (ChIP) or any combination thereof.

Transcriptomic biomarkers (such as RNA expression levels) may be detected by sequencing, microarrays, PCR, or any combination thereof.

### Classifier

A classifier algorithm may be used to garner insight into whether a biological sample evidences a presence, absence, or suspicion of cancer cells. The classifier algorithm may be used to analyze biomolecule information (e.g., DNA sequences, RNA sequences, and/or expression profiles) in samples that are otherwise inconclusive for cancer to determine whether the subject from which the sample was obtained has a pre-test high risk or pre-test low risk for cancer. As a non-limiting example, a bronchoscopy taken from a subject's lung nodule (initially detected via computerized tomography (CT) scan) may be determined to be inconclusive. Such a patient may be at a pre-test “intermediate” risk for lung cancer. Nasal swab samples may be taken from the subject and the nucleic acid molecules in these samples may be analyzed by sequencing to yield sequence information detect one or more genomic features. The classifier may be used to process the sequence information and down-classify the subject's sample (which may initially be inconclusive or intermediate risk) as post-test “low risk” for lung cancer or up-classify the subject as post-test “high-risk” for lung cancer.

For example, a pre-test risk of malignancy is low if it is less than or equal to about 10%, 9%, 8%, 7%, 6%, 5%, 4%, 3%, 2%, 1%, or less. A pre-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A pre-test risk of malignancy is intermediate if it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A pre-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

For example, a post-test risk of malignancy is low if it is less than or equal to about 10%, 9%, 8%, 7%, 6%, 5%, 4%, 3%, 2%, or 1%. A post-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A post-test risk of malignancy is intermediate it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A post-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

For example, post-test risk of malignancy is very low if it is less than about 1%, 0.9%, 0.8%, 0.7%, 0.6%, 0.5%, 0.4%, 0.3%, 0.2%, or 0.1%. A post-test risk of malignancy is low if less than about 10%, 9%, 8%, 7%, 6%, 5%, 4%, 3%, 2%, or 1.5%, and great than about 1%. A post-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A post-test risk of malignancy is intermediate it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A post-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, or 89%, and less than about 90%. A post-test risk of malignancy is very high if it is greater than about 90%, 91%, 92%, 93%, 94%, 95%, 96%, 97%, 98%, or 99%.

A classifier algorithm may be trained with one or more training samples. The classifier algorithm may be a trained algorithm (or trained machine learning algorithm). The one or more training samples may include covariates such as whether the sample was taken from an subject using inhaled medications, including for example bronchodilators, steroids, or a combination of bronchodilators and steroids, whether the sample was taken before or after a clinical sample, the smoking history of the subject, the gender of the subject, the current smoking status of the subject, etc. The classifier algorithm may be trained with a set of training samples that are independent of the sample analyzed by the classifier algorithm. The classifier algorithm may be trained with one or more different types of training samples. The classifier algorithm may be trained with at least two different types of training samples, such as a bronchial brushing sample and a fine needle aspiration. In another example, the training set may comprise samples benign for a lung condition and samples malignant for a lung condition. The training set may comprise samples that are determined to be benign for a lung condition and samples that are malignant for at least that same lung condition. A training data set may comprise samples obtained from subjects associated with a risk of developing lung cancer, examples include but are not limited to subjects with a history of smoking cigarettes or having an exposure to asbestos or having an exposure to air pollution (e.g., smog, smoke, etc.).

Training samples may be samples that are obtained from a subject prior to or following collection of a clinical sample (e.g., a biopsy or needle aspirate), or both. The training samples obtained before, after, or both before and after obtaining a clinical sample may be a nasal swab sample, a bronchial brushing sample, a buccal sample, or a bronchoscopy sample.

Training samples may include sample(s) that are from a subject(s) taking one or more inhaled medications. The inhaled medications may include, for example, bronchodilators, steroids, or a combination thereof. The sample may be obtained or provided after a clinical sample is extracted from the subject. The clinical sample may be a sample that is obtained by nasal swab, bronchial brushing, needle aspiration, or biopsy.

A classifier algorithm may be trained with at least three different types of training samples, such as a surgical biopsy, fine needle aspiration, buccal samples, and bronchial brushing. The classifier algorithm may be trained with at least three different types of training samples, such as a surgical biopsy, fine needle aspiration, swab, and bronchial brushing. The training samples can be correlated with an image obtained from a CT scan, X-ray or MRI. The classifier algorithm may be trained with at least four different types of training samples, such as a surgical biopsy, fine needle aspiration, swab, and bronchial brushing. The training samples can be correlated with an image obtained from a CT scan, X-ray or MRI. The classifier algorithm may be trained with bronchial brushing samples, buccal samples, and bronchoscopy samples labeled as normal, benign, cancerous, malignant, or any combination thereof. The samples may be labeled as cytologically normal or abnormal. The samples can be analyzed by histological analysis.

The methods and systems disclosed herein may classify a sample obtained from a subject as positive or negative for a lung condition (e.g., lung cancer) with high sensitivity, specificity, and/or accuracy. The sample may be classified as positive or negative for a lung condition (e.g., lung cancer) with a specificity of at least about 51%, 60% 70%, 80%, 85%, 90%, 95%, 99%, or greater. The sample may be classified as positive or negative for a lung condition (e.g., lung cancer) with a sensitivity of at least about 60% 70%, 80%, 85%, 90%, 95%, 99%, or greater. The sample may be classified as positive or negative for a lung condition (e.g., lung cancer) with an accuracy of at least about 60% 70%, 80%, 85%, 90%, 95%, 99%, or greater.

The methods and systems disclosed herein may determine that a subject has a likelihood of being free of a cancer. The subject may be determined to have a likelihood of at least about 50%, 70%, 80%, 90%, 95%, 99%, or greater of being free of a cancer.

Training samples used to train and validate a trained classifier algorithm may be greater than or equal to about: 100 samples, 200 samples, 300 samples, 400 samples, 500 samples, 600 samples, 700 samples, 800 samples, 900 samples, 1000 samples, 1100 samples, 1200 samples, 1300 samples, 1400 samples, 1500 samples, 1600 samples, 1700 samples, 1800 samples, 1900 samples, 2000 samples, or more (for example 1950 samples obtained from different subjects). In some cases, training samples may comprise from about 100 samples to about 200 samples. In some cases, training samples may comprise from about 100 samples to about 300 samples. In some cases, training samples may comprise from about 100 samples to about 400 samples. In some cases, training samples may comprise from about 100 samples to about 500 samples. In some cases, training samples may comprise from about 100 samples to about 600 samples. In some cases, training samples may comprise from about 100 samples to about 700 samples. In some cases, training samples may comprise from about 100 samples to about 800 samples. In some cases, training samples may comprise from about 100 samples to about 900 samples. In some cases, training samples may comprise from about 100 samples to about 1000 samples. In some cases, training samples may comprise from about 100 samples to about 1500 samples. In some cases, training samples may comprise from about 100 samples to about 2000 samples. In some cases, training samples may comprise from about 100 samples to about 3000 samples. In some cases, training samples may comprise from about 100 samples to about 4000 samples. In some cases, training samples may comprise from about 100 samples to about 5000 samples.

Training samples may be independent of the sample analyzed by the classifier algorithm. Training samples may be obtained from one or more subjects. Subject may include subjects having a different country of birth. Subject may include subject having a different place of residence. Training samples may represent at least about: 2, 3, 4, 5, 6, 7, 8, 9, 10, 11, 12, 13, 14, 15, 16, 17, 18, 19, or 20 different countries of birth. Training samples may represent at least about 3 different countries of birth. Training samples may represent at least about 5 different countries of birth. Training samples may represent at least about 10 different countries of birth. Training samples may represent from about 2 to about 10 different countries of birth. Training samples may represent from about 3 to about 15 different countries of birth. Training samples may represent from about 2 to about 20 different countries of birth. Training samples may represent at least about: 2, 3, 4, 5, 6, 7, 8, 9, 10, 11, 12, 13, 14, 15, 16, 17, 18, 19, or 20 different countries of residence. Training samples may represent at least about 3 different countries of residence. Training samples may represent at least about 5 different countries of residence. Training samples may represent at least about 10 different countries of residence. Training samples may represent from about 2 to about 10 different countries of residence. Training samples may represent from about 3 to about 15 different countries of residence. Training samples may represent from about 2 to about 20 different countries of residence.

Samples in the training set may comprise a plurality of conditions (such as diseases or disease subtypes, consumption of inhaled medication, timing of sample collection relative to clinical sample collection). Samples in an independent test (i.e., independent from the sample being assayed) set may comprise a plurality of conditions (such as disease or disease subtypes). Samples in an independent test set may comprise a least one disease or disease subtype that is different from the samples in the training set. Samples in the training set may comprise a least one disease or disease subtype that is different from the samples in the independent test set. Samples in the independent test set may comprise at least two additional diseases or disease subtypes than the samples in the training set.

Training samples may comprise one or more samples obtained from a subject suspected of having lung cancer, a subject having a confirmed diagnosis of lung cancer, a subject having a pre-existing condition such as a benign lung disease, a subject having lung nodules identified on a LDCT, a subject that may be a non-smoker, a subject that may be a non-smoker with environmental exposure to smoking, a current smoker, a previous smoker, a subject having smoked at least about: 1, 10, 20, 100, 200, 300, 400, 500, 600, 700, 800, 900, 1,000, 2,000, 3,000, 4,000, 5,000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000 or more cigarettes or cigars or e-cigarettes in their lifetime, a subject having an increased hereditary risk of developing lung cancer, a subject having a suppressed immune system, a subject having chronic pulmonary infections, or any combination thereof.

Intensity values or sequence information generated from nucleic acid sequencing for a sample may be analyzed using feature selection techniques including filter techniques which assess the relevance of features by looking at the intrinsic properties of the data, wrapper methods which embed the model hypothesis within a feature subset search, and embedded techniques in which the search for an optimal set of features may be built into a classifier algorithm.

Filter techniques that may be useful in the methods of the present disclosure include (1) parametric methods such as the use of two sample t-tests, ANOVA analyses, Bayesian frameworks, and Gamma distribution models (2) model free methods such as the use of Wilcoxon rank sum tests, between-within class sum of squares tests, rank products methods, random permutation methods, or TNoM which involves setting a threshold point for fold-change differences in expression between two datasets and then detecting the threshold point in each gene that minimizes the number of misclassifications (3) and multivariate methods such as bivariate methods, correlation based feature selection methods (CFS), minimum redundancy maximum relevance methods (MRMR), Markov blanket filter methods, and uncorrelated shrunken centroid methods. Wrapper methods useful in the methods of the present disclosure include sequential search methods, genetic algorithms, and estimation of distribution algorithms. Embedded methods useful in the methods of the present disclosure include random forest algorithms, weight vector of support vector machine algorithms, and weights of logistic regression algorithms. Bioinformatics, 2007 Oct. 1; 23(19):2507-17 provides an overview of the relative merits of the filter techniques provided above for the analysis of intensity data.

### Clinical Covariates

The classifier can comprise clinical covariates. Clinical covariates can include age, nodule length (log2 transformed), nodule spiculation (Y/N), pack-year, genomic gender, genomic smoking duration index, or genomic smoking status (current vs. former) index. Clinical covariates can comprise radiographic features such as nodule spiculation and nodule length. Genomic indexes for gender, smoking status, and smoking burden are disclosed herein. As blood contamination can impact classifier performance, Hemoglobin Subunit Beta gene expression can be used to measure a degree of contamination as a prospective exclusion criterion.

The one or more genomic index can comprise a genomic gender index. The genomic gender index can comprise one or more of USP9Y, RPS4Y1, UTY, DDX3Y, or KDM5D.

Pack years can be less than 20 packs, between 20 and 50 packs, or greater than 50 packs. Pack years may correlate to an individual having at least about: 1, 5, 10, 20, 30, 40, 50, 60, 70, 80, 90, 100, 200, 300, 400, or 500 cigarettes, cigars, or e-cigarettes in their lifetime. An individual may have had at least about 100 cigarettes, cigars, or e-cigarettes in their lifetime. A smoker may be an individual having at least about 500 cigarettes, cigars, or e-cigarettes in their lifetime. A smoker may be an individual having had greater than about: 5, 10, 20, 30, 40, or 50 packs of cigarettes, cigars, e-cigarettes per year. A smoker may be an individual having had greater than about 5 packs of cigarettes, cigars, e-cigarettes per year. A smoker may be an individual having had greater than about 10 packs of cigarettes, cigars, e-cigarettes per year. A smoker may be an individual having had greater than about 20 packs of cigarettes, cigars, e-cigarettes per year. A smoker may be an individual having had greater than about 30 packs of cigarettes, cigars, e-cigarettes per year. A smoker may be an individual having had from about 1 pack to about 12 packs (or more) of cigarettes, cigars, e-cigarettes per year. A smoker may be an individual having had from about 10 packs to about 25 packs of cigarettes, cigars, e-cigarettes per year. A smoker may be an individual having had from about 25 packs to about 50 packs of cigarettes, cigars, e-cigarettes per year. A smoker may be an individual having had from about 1 pack to about 50 packs of cigarettes, cigars, e-cigarettes per year. A smoker may be an individual having had from about 10 packs to about 50 packs of cigarettes, cigars, e-cigarettes per year.

The genomic smoking status index can comprise the evaluation of an expression level of one or more genes from Table 1. The genomic smoking status index can comprise the evaluation of an expression level of less than or equal to 80, 70, 60, 50, 40, 30, 20, 19, 18, 17, 16, 15, 14, 13, 12, 11, 10, 9, 8, 7, 6, 5, 4, 3, or 2 genes. The genomic smoking status index can comprise the evaluation of an expression level of greater than or equal to 1, 2, 3, 4, 5, 6, 7, 8, 9, 10, 11, 12, 13, 14, 15, 16, 17, 18, 19, 20, 30, 40, 50, 60, 70, or 80 genes. The one or more genes can be selected from: ACVRL1, AHRR, AP1S3, ARRDC4, B3GNT6, BAALC, BPIFB2, CACNA2D3, CCDC69, CCDC88A, CD163L1, CDK5RAP2, CIT, CLIC5, CMTM7, CNGB1, COL1A2, COL3A1, COL6A3, CPE, CPNE8, CRNN, CYP2A13, CYP4X1, EDC3, ENC1, ENTPD8, FHL1, FOXE1, GAD1, GLDN, GLYATL2, GRAMD2, GSTO2, hsa-mir-7162, HSF4, ICA1, IGF1, IL36A, JAKMIP3, KPRP, LCE3D, LRRC31, MAMDC2, MGP, MMP7, MPST, NOL3, NOX4, NRIP1, OCA2, PANX2, PBX3, PRKAR2B, RAMP1, RDH10, RHCG, RNF175, RPTN, SAA1, SAA2, SAMHD1, SERPINE2, SETD7, SLC16A12, SLC28A2, SLPI, TGM3, TGM6, TIPARP, TMEM45B, TRHDE, TRNAU1AP, UCHL1, USH1C, USP54, WNTSA, or ZKSCAN1.

Radiographic features disclosed herein can include nodule length and nodule spiculation. A nodule length can be less than 6 mm, between 6 mm and 30 mm, greater than 30 mm, or less than 4 mm. Nodule spiculation can be described as the appearance of a “corona radiata” or “sunburst” like border around a nodule identified by imaging analysis.

The classifier can comprise one or more genomic index. The genomic index can comprise genes associated with one or more genomic covariates. Genomic covariates can include gender, smoking duration, smoking status (current v. former), cell type, and genes associated with noise (batch genes). The genomic index can be used to separate a benign or malignant expression profile from noise (signal not associated with whether a sample is from a subject with a benign or malignant nodule). The genomic index can be used to identify the cell types in a sample. The genomic index can be used to determine the smoking status of an individual, for example whether the individual is a current or former smoker.

The genomic smoking duration index can be used to determine how long an individual has been exposed to smoke. Smoking duration can be less than 1 year, between 2 and 10 years, or greater than 10 years. Smoking duration may correlate to an individual smoking for at least about: 1, 5, 10, 20, 30, 40, 50, or 60 years. Smoking duration may correlate to an individual smoking for less than about: 50, 40, 30, 20, 10, 5, or 1 year. The genomic smoking duration index can comprise the evaluation of an expression level of one or more genes from Table 1. The genomic smoking duration index can comprise the evaluation of an expression level of less than or equal to 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, or 2 genes. The genomic smoking duration index can comprise the evaluation of an expression level of greater than or equal to 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, or 190 genes. The one or more genes can be selected from AC074091.1, ACTL10, ADRA2B, AGT, ALDOC, AMACR, AOX1, APEH, APOPT1, ARHGEF35, ARNTL, ATF7IP2, ATP2A3, BBOX1, BHLHE40-AS1, BNIP3, BOLA1, BPI, C11orf68, C12orf65, C1QL2, C21orf128, C2orf73, CACNA1B, CAPG, CAPN9, CDC25A, CDC42P6, CDCA2, CDCP1, CDHR1, CDHR2, CDK5, CDNF, CMTM2, COG1, COL1A1, COL5A3, CORO2B, CST7, CTD-2555O16.2, CTD-2555O16.4, CTGLF12P, CTNS, CTSF, CXCL12, CYP7B1, DBI, DDO, DDT, DLL1, DOCK3, DRD4, EDIL3, EFHB, ETFDH, EVA1A, FAM184A, FAM189B, FLT1, FOXC2, FTCDNL1, GALNT16, GET4, GLB1L3, GNAL, GNG4, GOLGA8O, GOT1, HARBI1, HAUS4, HCAR3, HERC2P2, HIST1H3E, HIST1H4F, HLA-J, HORMAD1, HSF4, HSF5, IGF2BP2, ISYNA1, KCNMB3, KCNQ3, KCTD10, KDR, KIAA0513, KRT39, KRT40, KRTAP5-7, LOXHD1, LTBP1, LUZP2, LYRM5, MAD2L1BP, MMD, MMP1, MPP7, MRM1, MRPS6, MRVI1-AS1, MUC6, MUT, MVB12A, NAMPTL, NBR2, NDUFA6, NDUFAF6, NDUFS7, NEFH, NLRP2, NME6, NPSR1, NUDT7, OLFM1, ORAOV1, PALM3, PAPSS1, PCDHA12, PCDHA13, PCDHB11, PCDHB16, PDPR, PEX11A, PIAS2, PIPOX, PLAG1, PLG, PMP22, PMS2P5, POLR2M, PPFIA3, PPP1R42, PRPF38B, PTGER4, RANGRF, RBMS3, RIMBP2, RIMKLA, RND2, RP11-163E9.2, RP11-171I2.2, RP11-171I2.4, RP11-345J4.8, RP11-461A8.1, RP11-477D19.2, RP11-522I20.3, RP11-695J4.2, RPL9, RUSC1, SCN11A, SDHAF2, SEMA3F, SEPT7P9, SFRP2, SH3GL3, SLAMF6, SLC22A3, SLC37A2, SLC48A1, SLC6A13, SNORD101, SP6, SPINK1, STAG3L2, STXBP5L, TEKT4, TERF2, TF, TFAP2C, TMEM200C, TMEM213, TMTC4, TP53I11, TTC39B, TTLL13, TWF2, TYRO3, UBAP1L, WDR53, WIPF3, ZFP2, ZFP28, ZNF232, ZNF576, or ZNF624.

Selected features may then be classified using a classifier algorithm. Illustrative algorithms include but may not be limited to methods that reduce the number of variables such as principal component analysis algorithms, partial least squares methods, and independent component analysis algorithms. Illustrative algorithms further include but may not be limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques may include bagging procedures, boosting procedures, random forest algorithms, and combinations thereof. See, e.g., Cancer Inform, 2008; 6:77-97 , Clin Transl. Sci., 2011; 4(6):466-477, and J.Phys.Conf.Ser., 2018;971, which is entirely incorporated herein by reference, and J. Proteomics Bioinform., 2010; 3(6):183-190, which is entirely incorporated herein by reference.

Systems and methods of the present disclosure may enable 1) gene expression analysis of a sample containing low amounts and/or low quality of nucleic acids; 2) a significant reduction of false positives and false negatives, 3) a determination of the underlying genetic, metabolic, or signaling pathways responsible for the resulting pathology, 4) the ability to assign a statistical probability to the accuracy of a diagnosis, a risk of developing a condition, a monitoring of changes in a condition, an effectiveness of an interventive therapy, or combinations thereof, 5) the ability to resolve ambiguous results, and 6) the ability to distinguish between lung conditions or sub-types of lung conditions based on the presence of a plurality of genomic and/or clinical features.

A sample may be contaminated with blood. For example, the sample may contain less than 1%, less than 5%, less than 10%, less than 20%, less than 30%, less than 40%, or less than 50% blood content. A sample can contain more than 1%, more than 5%, more than 10%, more than 20%, more than 30%, or more than 40% blood content.

A sample may contain a low amount of nucleic acids. For example, the sample may contain less than 100 picograms (pg) of DNA, less than 90 pg of DNA, less than 80 pg of DNA, less than 70 pg of DNA, less than 60 pg of DNA, less than 50 pg of DNA, less than 40 pg of DNA, less than 30 pg of DNA, less than 20 pg of DNA, less than 10 pg of DNA. A samples may contain more than 100 pg of DNA, more than 90 pg of DNA, more than 80 pg of DNA, more than 70 pg of DNA, more than 60 pg of DNA, more than 50 pg of DNA, more than 40 pg of DNA, more than 30 pg of DNA, more than 20 pg of DNA, more than 10pg of DNA. A sample may contain less than 60 nanograms (ng) of RNA, less than 50 ng of RNA, less than 40 ng of RNA, less than 30 ng of RNA, less than 20 ng of RNA, less than 10 ng of RNA, less than 5 ng of RNA. A sample may contain more than 60 ng of RNA, 50 ng of RNA, 40 ng of RNA, 30 ng of RNA, 20 ng of RNA, 10 ng of RNA, 5 ng of RNA. The sample may contain nucleic acids that are of low quality (e.g., as determined by RNA integrity number). Low quality nucleic acid molecules comprising RNA may have an RNA integrity number (“RIN”) of less than 5.0, less than 4.5, less than 4.0, less than 3.5, less than 3.0, less than 2.5, less than 2.0, less than 1.5. Low quality nucleic acid molecules comprising RNA may have a RIN of less than 3.0.

Methods disclosed herein can comprise the measurement of the expression of one or more genes correlated with a risk of lung cancer. The one or more genes can be selected from the 502 genes listed in Table 1. Methods disclosed herein can comprise the evaluation of an expression level of greater than or equal to 1, 2, 3, 4, 5, 6, 7, 8, 9 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 genes selected from Table 1. Methods disclosed herein can comprise the evaluation of an expression level of less than or equal to 502, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, or 2 genes selected from Table 1. Methods disclosed herein can comprise the evaluation of an expression level of between 1 and 10, 5 and 25, 20 and 50, 30 and 100, 60 and 150, 70 and 200, 100 and 300, 200 and 400, or 300 and 500 genes selected from Table 1.

TABLE 1
502 Classifier Genes

| ENSG Ref. | Gene Name | Genomic Index |
|---|---|---|
| ENSG00000183044 | ABAT | benign/malignant (“BM”) |
| ENSG00000069431 | ABCC9 | BM |
| ENSG00000097007 | ABL1 | BM |
| ENSG00000143322 | ABL2 | BM |
| ENSG00000221531 | AC074091.1 | smoking duration |
| ENSG00000182584 | ACTL10 | smoking duration |
| ENSG00000139567 | ACVRL1 | smoking status |
| ENSG00000143537 | ADAM15 | BM |
| ENSG00000008277 | ADAM22 | BM |
| ENSG00000197140 | ADAM32 | BM |
| ENSG00000154734 | ADAMTS1 | BM |
| ENSG00000222040 | ADRA2B | smoking duration |
| ENSG00000135744 | AGT | smoking duration |
| ENSG00000158467 | AHCYL2 | BM |
| ENSG00000063438 | AHRR | smoking status |
| ENSG00000109107 | ALDOC | smoking duration |
| ENSG00000242110 | AMACR | smoking duration |
| ENSG00000151743 | AMN1 | BM |
| ENSG00000145362 | ANK2 | BM |
| ENSG00000138356 | AOX1 | BM; smoking duration |
| ENSG00000152056 | AP1S3 | smoking status |
| ENSG00000164062 | APEH | smoking duration |
| ENSG00000256053 | APOPT1 | smoking duration |
| ENSG00000165272 | AQP3 | BM |
| ENSG00000213214 | ARHGEF35 | smoking duration |
| ENSG00000122644 | ARL4A | BM |
| ENSG00000133794 | ARNTL | smoking duration |
| ENSG00000140450 | ARRDC4 | smoking status |
| ENSG00000004848 | ARX | BM |
| ENSG00000128203 | ASPHD2 | BM |
| ENSG00000166669 | ATF7IP2 | smoking duration |
| ENSG00000074370 | ATP2A3 | smoking duration |
| ENSG00000113732 | ATP6V0E1 | BM |
| ENSG00000162779 | AXDND1 | BM |
| ENSG00000198488 | B3GNT6 | smoking status |
| ENSG00000164929 | BAALC | smoking status |
| ENSG00000129151 | BBOX1 | smoking duration |
| ENSG00000075790 | BCAP29 | BM |
| ENSG00000235831 | BHLHE40-AS1 | smoking duration |
| ENSG00000100290 | BIK | BM |
| ENSG00000152785 | BMP3 | BM |
| ENSG00000176171 | BNIP3 | BM; smoking duration |
| ENSG00000104765 | BNIP3L | BM |
| ENSG00000178096 | BOLA1 | smoking duration |
| ENSG00000101425 | BPI | smoking duration |
| ENSG00000078898 | BPIFB2 | smoking status |
| ENSG00000164713 | BRI3 | BM |
| ENSG00000175573 | C11orf68 | smoking duration |
| ENSG00000130921 | C12orf65 | smoking duration |
| ENSG00000186960 | C14orf23 | BM |
| ENSG00000144119 | C1QL2 | smoking duration |
| ENSG00000184385 | C21orf128 | smoking duration |
| ENSG00000111731 | C2CD5 | BM |
| ENSG00000177994 | C2orf73 | smoking duration |
| ENSG00000186577 | C6orf1 | BM |
| ENSG00000148408 | CACNA1B | BM; smoking duration |
| ENSG00000157445 | CACNA2D3 | smoking status |
| ENSG00000042493 | CAPG | smoking duration |
| ENSG00000135773 | CAPN9 | smoking duration |
| ENSG00000147044 | CASK | BM |
| ENSG00000174898 | CATSPERD | BM |
| ENSG00000198624 | CCDC69 | smoking status |
| ENSG00000091986 | CCDC80 | BM |
| ENSG00000115355 | CCDC88A | smoking status |
| ENSG00000129315 | CCNT1 | BM |
| ENSG00000177675 | CD163L1 | smoking status |
| ENSG00000091972 | CD200 | BM |
| ENSG00000164045 | CDC25A | smoking duration |
| ENSG00000237350 | CDC42P6 | smoking duration |
| ENSG00000184661 | CDCA2 | smoking duration |
| ENSG00000163814 | CDCP1 | smoking duration |
| ENSG00000148600 | CDHR1 | BM; smoking duration |
| ENSG00000074276 | CDHR2 | smoking duration |
| ENSG00000164885 | CDK5 | smoking duration |
| ENSG00000136861 | CDK5RAP2 | smoking status |
| ENSG00000185267 | CDNF | smoking duration |
| ENSG00000197766 | CFD | BM |
| ENSG00000170791 | CHCHD7 | BM |
| ENSG00000122966 | CIT | smoking status |
| ENSG00000164442 | CITED2 | BM |
| ENSG00000186510 | CLCNKA | BM |
| ENSG00000112782 | CLIC5 | smoking status |
| ENSG00000074201 | CLNS1A | BM |
| ENSG00000162368 | CMPK1 | BM |
| ENSG00000140932 | CMTM2 | smoking duration |
| ENSG00000153551 | CMTM7 | smoking status |
| ENSG00000144191 | CNGA3 | BM |
| ENSG00000070729 | CNGB1 | smoking status |
| ENSG00000162852 | CNST | BM |
| ENSG00000144619 | CNTN4 | BM |
| ENSG00000068120 | COASY | BM |
| ENSG00000166685 | COG1 | smoking duration |
| ENSG00000108821 | COL1A1 | smoking duration |
| ENSG00000164692 | COL1A2 | smoking status |
| ENSG00000168542 | COL3A1 | smoking status |
| ENSG00000080573 | COL5A3 | smoking duration |
| ENSG00000142156 | COL6A1 | BM |
| ENSG00000163359 | COL6A3 | smoking status |
| ENSG00000110880 | CORO1C | BM |
| ENSG00000103647 | CORO2B | smoking duration |
| ENSG00000109472 | CPE | smoking status |
| ENSG00000139117 | CPNE8 | smoking status |
| ENSG00000095321 | CRAT | BM |
| ENSG00000134376 | CRB1 | BM |
| ENSG00000096006 | CRISP3 | BM |
| ENSG00000121005 | CRISPLD1 | BM |
| ENSG00000143536 | CRNN | smoking status |
| ENSG00000121904 | CSMD2 | BM |
| ENSG00000170373 | CST1 | BM |
| ENSG00000077984 | CST7 | smoking duration |
| ENSG00000258824 | CTD-2555O16.2 | smoking duration |
| ENSG00000272909 | CTD-2555O16.4 | smoking duration |
| ENSG00000179296 | CTGLF12P | smoking duration |
| ENSG00000040531 | CTNS | smoking duration |
| ENSG00000174080 | CTSF | smoking duration |
| ENSG00000085733 | CTTN | BM |
| ENSG00000107611 | CUBN | BM |
| ENSG00000180891 | CUEDC1 | BM |
| ENSG00000107562 | CXCL12 | smoking duration |
| ENSG00000197838 | CYP2A13 | smoking status |
| ENSG00000186377 | CYP4X1 | smoking status |
| ENSG00000172817 | CYP7B1 | smoking duration |
| ENSG00000123977 | DAW1 | BM |
| ENSG00000155368 | DBI | smoking duration |
| ENSG00000003249 | DBNDD1 | BM |
| ENSG00000136485 | DCAF7 | BM |
| ENSG00000203797 | DDO | smoking duration |
| ENSG00000244038 | DDOST | BM |
| ENSG00000204580 | DDR1 | BM |
| ENSG00000099977 | DDT | smoking duration |
| ENSG00000067048 | DDX3Y | gender |
| ENSG00000065357 | DGKA | BM |
| ENSG00000198719 | DLL1 | smoking duration |
| ENSG00000116675 | DNAJC6 | BM |
| ENSG00000088538 | DOCK3 | BM; smoking duration |
| ENSG00000069696 | DRD4 | smoking duration |
| ENSG00000161326 | DUSP14 | BM |
| ENSG00000141627 | DYM | BM |
| ENSG00000127884 | ECHS1 | BM |
| ENSG00000179151 | EDC3 | smoking status |
| ENSG00000164176 | EDIL3 | smoking duration |
| ENSG00000163576 | EFHB | smoking duration |
| ENSG00000179387 | ELMOD2 | BM |
| ENSG00000132464 | ENAM | BM |
| ENSG00000171617 | ENC1 | smoking status |
| ENSG00000120658 | ENOX1 | BM |
| ENSG00000112796 | ENPP5 | BM |
| ENSG00000188833 | ENTPD8 | smoking status |
| ENSG00000146904 | EPHA1 | BM |
| ENSG00000103067 | ESRP2 | BM |
| ENSG00000171503 | ETFDH | smoking duration |
| ENSG00000115363 | EVA1A | smoking duration |
| ENSG00000198420 | FAM115A | BM |
| ENSG00000121104 | FAM117A | BM |
| ENSG00000111879 | FAM184A | smoking duration |
| ENSG00000160767 | FAM189B | smoking duration |
| ENSG00000198643 | FAM3D | BM |
| ENSG00000005812 | FBXL3 | BM |
| ENSG00000138081 | FBXO11 | BM |
| ENSG00000142748 | FCN3 | BM |
| ENSG00000137714 | FDX1 | BM |
| ENSG00000022267 | FHL1 | smoking status |
| ENSG00000100442 | FKBP3 | BM |
| ENSG00000154803 | FLCN | BM |
| ENSG00000102755 | FLT1 | smoking duration |
| ENSG00000217128 | FNIP1 | BM |
| ENSG00000052795 | FNIP2 | BM |
| ENSG00000176692 | FOXC2 | smoking duration |
| ENSG00000178919 | FOXE1 | smoking status |
| ENSG00000137166 | FOXP4 | BM |
| ENSG00000169933 | FRMPD4 | BM |
| ENSG00000226124 | FTCDNL1 | smoking duration |
| ENSG00000128683 | GAD1 | smoking status |
| ENSG00000100626 | GALNT16 | smoking duration |
| ENSG00000143641 | GALNT2 | BM |
| ENSG00000164949 | GEM | BM |
| ENSG00000239857 | GET4 | smoking duration |
| ENSG00000151892 | GFRA1 | BM |
| ENSG00000166105 | GLB1L3 | smoking duration |
| ENSG00000186417 | GLDN | smoking status |
| ENSG00000107249 | GLIS3 | BM |
| ENSG00000156689 | GLYATL2 | smoking status |
| ENSG00000141404 | GNAL | smoking duration |
| ENSG00000168243 | GNG4 | smoking duration |
| ENSG00000124713 | GNMT | BM |
| ENSG00000147437 | GNRH1 | BM |
| ENSG00000215186 | GOLGA6B | BM |
| ENSG00000206127 | GOLGA8O | smoking duration |
| ENSG00000120053 | GOT1 | smoking duration |
| ENSG00000069122 | GPR116 | BM |
| ENSG00000175697 | GPR156 | BM |
| ENSG00000167191 | GPRC5B | BM |
| ENSG00000175318 | GRAMD2 | smoking status |
| ENSG00000158055 | GRHL3 | BM |
| ENSG00000065621 | GSTO2 | smoking status |
| ENSG00000111713 | GYS2 | BM |
| ENSG00000130600 | H19 | BM |
| ENSG00000180423 | HARBI1 | smoking duration |
| ENSG00000092036 | HAUS4 | smoking duration |
| ENSG00000255398 | HCAR3 | smoking duration |
| ENSG00000101336 | HCK | BM |
| ENSG00000162639 | HENMT1 | BM |
| ENSG00000140181 | HERC2P2 | smoking duration |
| ENSG00000188290 | HES4 | BM |
| ENSG00000196966 | HIST1H3E | smoking duration |
| ENSG00000198327 | HIST1H4F | smoking duration |
| ENSG00000204622 | HLA-J | smoking duration |
| ENSG00000143452 | HORMAD1 | smoking duration |
| ENSG00000158104 | HPD | BM |
| ENSG00000166104 | hsa-mir-7162 | smoking status |
| ENSG00000086696 | HSD17B2 | BM |
| ENSG00000102878 | HSF4 | smoking status; smoking |
| | | duration |
| ENSG00000176160 | HSF5 | smoking duration |
| ENSG00000102241 | HTATSF1 | BM |
| ENSG00000003147 | ICA1 | smoking status |
| ENSG00000162783 | IER5 | BM |
| ENSG00000006652 | IFRD1 | BM |
| ENSG00000017427 | IGF1 | smoking status |
| ENSG00000073792 | IGF2BP2 | smoking duration |
| ENSG00000142677 | IL22RA1 | BM |
| ENSG00000136694 | IL36A | smoking status |
| ENSG00000151689 | INPP1 | BM |
| ENSG00000185085 | INTS5 | BM |
| ENSG00000105655 | ISYNA1 | smoking duration |
| ENSG00000188385 | JAKMIP3 | smoking status |
| ENSG00000166086 | JAM3 | BM |
| ENSG00000136504 | KAT7 | BM |
| ENSG00000171121 | KCNMB3 | smoking duration |
| ENSG00000184156 | KCNQ3 | BM; smoking duration |
| ENSG00000110906 | KCTD10 | smoking duration |
| ENSG00000012817 | KDM5D | gender |
| ENSG00000128052 | KDR | smoking duration |
| ENSG00000112232 | KHDRBS2 | BM |
| ENSG00000135709 | KIAA0513 | smoking duration |
| ENSG00000165757 | KIAA1462 | BM |
| ENSG00000129250 | KIF1C | BM |
| ENSG00000162413 | KLHL21 | BM |
| ENSG00000239474 | KLHL41 | BM |
| ENSG00000203786 | KPRP | smoking status |
| ENSG00000196859 | KRT39 | smoking duration |
| ENSG00000204889 | KRT40 | smoking duration |
| ENSG00000205426 | KRT81 | BM |
| ENSG00000170442 | KRT86 | BM |
| ENSG00000244411 | KRTAP5-7 | smoking duration |
| ENSG00000149357 | LAMTOR1 | BM |
| ENSG00000150457 | LATS2 | BM |
| ENSG00000163202 | LCE3D | smoking status |
| ENSG00000174106 | LEMD3 | BM |
| ENSG00000166477 | LEO1 | BM |
| ENSG00000168924 | LETM1 | BM |
| ENSG00000167210 | LOXHD1 | smoking duration |
| ENSG00000134324 | LPIN1 | BM |
| ENSG00000010626 | LRRC23 | BM |
| ENSG00000114248 | LRRC31 | smoking status |
| ENSG00000185158 | LRRC37B | BM |
| ENSG00000049323 | LTBP1 | smoking duration |
| ENSG00000187398 | LUZP2 | smoking duration |
| ENSG00000205707 | LYRM5 | smoking duration |
| ENSG00000124688 | MAD2L1BP | smoking duration |
| ENSG00000165072 | MAMDC2 | smoking status |
| ENSG00000131711 | MAP1B | BM |
| ENSG00000124641 | MED20 | smoking status |
| ENSG00000010165 | METTL13 | BM |
| ENSG00000074416 | MGLL | BM |
| ENSG00000111341 | MGP | BM; smoking status |
| ENSG00000199072 | MIRLET7F1 | BM |
| ENSG00000108960 | MMD | smoking duration |
| ENSG00000196611 | MMP1 | smoking duration |
| ENSG00000137745 | MMP13 | BM |
| ENSG00000137673 | MMP7 | smoking status |
| ENSG00000107186 | MPDZ | BM |
| ENSG00000150054 | MPP7 | smoking duration |
| ENSG00000128309 | MPST | smoking status |
| ENSG00000129282 | MRM1 | smoking duration |
| ENSG00000117501 | MROH9 | smoking status |
| ENSG00000243927 | MRPS6 | smoking duration |
| ENSG00000177112 | MRVI1-AS1 | smoking duration |
| ENSG00000132938 | MTUS2 | BM |
| ENSG00000184956 | MUC6 | BM; smoking duration |
| ENSG00000171195 | MUC7 | BM |
| ENSG00000146085 | MUT | smoking duration |
| ENSG00000141971 | MVB12A | smoking duration |
| ENSG00000013364 | MVP | BM |
| ENSG00000170011 | MYRIP | BM |
| ENSG00000102030 | NAA10 | BM |
| ENSG00000128534 | NAA38 | BM |
| ENSG00000229644 | NAMPTL | smoking duration |
| ENSG00000168614 | NBPF9 | BM |
| ENSG00000198496 | NBR2 | smoking duration |
| ENSG00000149294 | NCAM1 | BM |
| ENSG00000103034 | NDRG4 | BM |
| ENSG00000184983 | NDUFA6 | smoking duration |
| ENSG00000156170 | NDUFAF6 | smoking duration |
| ENSG00000213619 | NDUFS3 | BM |
| ENSG00000115286 | NDUFS7 | smoking duration |
| ENSG00000167792 | NDUFV1 | BM |
| ENSG00000129559 | NEDD8 | BM |
| ENSG00000100285 | NEFH | smoking duration |
| ENSG00000172260 | NEGR1 | BM |
| ENSG00000022556 | NLRP2 | smoking duration |
| ENSG00000172113 | NME6 | smoking duration |
| ENSG00000184967 | NOC4L | BM |
| ENSG00000140939 | NOL3 | smoking status |
| ENSG00000139910 | NOVA1 | BM |
| ENSG00000086991 | NOX4 | smoking status |
| ENSG00000151322 | NPAS3 | BM |
| ENSG00000187258 | NPSR1 | smoking duration |
| ENSG00000180530 | NRIP1 | smoking status |
| ENSG00000140876 | NUDT7 | smoking duration |
| ENSG00000104044 | OCA2 | smoking status |
| ENSG00000130558 | OLFM1 | smoking duration |
| ENSG00000149716 | ORAOV1 | smoking duration |
| ENSG00000187867 | PALM3 | smoking duration |
| ENSG00000073150 | PANX2 | smoking status |
| ENSG00000182752 | PAPPA | BM |
| ENSG00000138801 | PAPSS1 | smoking duration |
| ENSG00000227345 | PARG | BM |
| ENSG00000198807 | PAX9 | BM |
| ENSG00000167081 | PBX3 | smoking status |
| ENSG00000251664 | PCDHA12 | smoking duration |
| ENSG00000239389 | PCDHA13 | smoking duration |
| ENSG00000197479 | PCDHB11 | smoking duration |
| ENSG00000196963 | PCDHB16 | smoking duration |
| ENSG00000128655 | PDE11A | BM |
| ENSG00000107438 | PDLIM1 | BM |
| ENSG00000090857 | PDPR | smoking duration |
| ENSG00000166821 | PEX11A | smoking duration |
| ENSG00000141959 | PFKL | BM |
| ENSG00000033800 | PIAS1 | BM |
| ENSG00000078043 | PIAS2 | smoking duration |
| ENSG00000143398 | PIP5K1A | BM |
| ENSG00000179761 | PIPOX | smoking duration |
| ENSG00000181690 | PLAG1 | smoking duration |
| ENSG00000153404 | PLEKHG4B | BM |
| ENSG00000225190 | PLEKHM1 | BM |
| ENSG00000122194 | PLG | smoking duration |
| ENSG00000109099 | PMP22 | smoking duration |
| ENSG00000123965 | PMS2P5 | smoking duration |
| ENSG00000255529 | POLR2M | smoking duration |
| ENSG00000013503 | POLR3B | BM |
| ENSG00000177380 | PPFIA3 | smoking duration |
| ENSG00000168938 | PPIC | BM |
| ENSG00000178125 | PPP1R42 | smoking duration |
| ENSG00000112640 | PPP2R5D | BM |
| ENSG00000116731 | PRDM2 | BM |
| ENSG00000005249 | PRKAR2B | BM; smoking status |
| ENSG00000184304 | PRKD1 | BM |
| ENSG00000134186 | PRPF38B | smoking duration |
| ENSG00000204576 | PRR3 | BM |
| ENSG00000171522 | PTGER4 | smoking duration |
| ENSG00000080031 | PTPRH | BM |
| ENSG00000172053 | QARS | BM |
| ENSG00000132155 | RAF1 | BM |
| ENSG00000108557 | RAI1 | BM |
| ENSG00000132329 | RAMP1 | smoking status |
| ENSG00000108961 | RANGRF | smoking duration |
| ENSG00000113319 | RASGRF2 | BM |
| ENSG00000122257 | RBBP6 | BM |
| ENSG00000144642 | RBMS3 | smoking duration |
| ENSG00000121039 | RDH10 | smoking status |
| ENSG00000135597 | REPS1 | BM |
| ENSG00000158315 | RHBDL2 | BM |
| ENSG00000140519 | RHCG | smoking status |
| ENSG00000126858 | RHOT1 | BM |
| ENSG00000060709 | RIMBP2 | smoking duration |
| ENSG00000177181 | RIMKLA | smoking duration |
| ENSG00000117000 | RLF | BM |
| ENSG00000137824 | RMDN3 | BM |
| ENSG00000219200 | RNASEK | BM |
| ENSG00000108830 | RND2 | smoking duration |
| ENSG00000166439 | RNF169 | BM |
| ENSG00000145428 | RNF175 | smoking status |
| ENSG00000138942 | RNF185 | BM |
| ENSG00000239969 | RP11-163E9.2 | smoking duration |
| ENSG00000270574 | RP11-171I2.2 | smoking duration |
| ENSG00000271141 | RP11-171I2.4 | smoking duration |
| ENSG00000205534 | RP11-345J4.8 | smoking duration |
| ENSG00000261938 | RP11-461A8.1 | smoking duration |
| ENSG00000235381 | RP11-477D19.2 | smoking duration |
| ENSG00000254473 | RP11-522I20.3 | smoking duration |
| ENSG00000256751 | RP11-695J4.2 | smoking duration |
| ENSG00000116745 | RPE65 | BM |
| ENSG00000163682 | RPL9 | smoking duration |
| ENSG00000129824 | RPS4Y1 | gender |
| ENSG00000215853 | RPTN | smoking status |
| ENSG00000144580 | RQCD1 | BM |
| ENSG00000160753 | RUSC1 | smoking duration |
| ENSG00000198853 | RUSC2 | BM |
| ENSG00000163602 | RYBP | BM |
| ENSG00000189171 | S100A13 | BM |
| ENSG00000173432 | SAA1 | smoking status |
| ENSG00000134339 | SAA2 | smoking status |
| ENSG00000156671 | SAMD8 | BM |
| ENSG00000101347 | SAMHD1 | smoking status |
| ENSG00000244486 | SCARF2 | BM |
| ENSG00000251992 | SCARNA17 | BM |
| ENSG00000168356 | SCN11A | BM; smoking duration |
| ENSG00000146197 | SCUBE3 | BM |
| ENSG00000167985 | SDHAF2 | smoking duration |
| ENSG00000214491 | SEC14L6 | BM |
| ENSG00000138802 | SEC24B | BM |
| ENSG00000001617 | SEMA3F | smoking duration |
| ENSG00000095539 | SEMA4G | BM |
| ENSG00000120555 | SEPT7P9 | smoking duration |
| ENSG00000135919 | SERPINE2 | smoking status |
| ENSG00000145391 | SETD7 | smoking status |
| ENSG00000145423 | SFRP2 | smoking duration |
| ENSG00000140600 | SH3GL3 | smoking duration |
| ENSG00000162105 | SHANK2 | BM |
| ENSG00000196470 | SIAH1 | BM |
| ENSG00000109171 | SLAIN2 | BM |
| ENSG00000162739 | SLAMF6 | smoking duration |
| ENSG00000152779 | SLC16A12 | smoking status |
| ENSG00000117479 | SLC19A2 | BM |
| ENSG00000168575 | SLC20A2 | BM |
| ENSG00000146477 | SLC22A3 | smoking duration |
| ENSG00000170482 | SLC23A1 | BM |
| ENSG00000137860 | SLC28A2 | smoking status |
| ENSG00000134955 | SLC37A2 | smoking duration |
| ENSG00000211584 | SLC48A1 | smoking duration |
| ENSG00000163959 | SLC51A | BM |
| ENSG00000010379 | SLC6A13 | smoking duration |
| ENSG00000124107 | SLPI | smoking status |
| ENSG00000073584 | SMARCE1 | BM |
| ENSG00000145335 | SNCA | BM |
| ENSG00000159210 | SNF8 | BM |
| ENSG00000206754 | SNORD101 | smoking duration |
| ENSG00000222365 | SNORD12B | BM |
| ENSG00000060688 | SNRNP40 | BM |
| ENSG00000174226 | SNX31 | BM |
| ENSG00000198142 | SOWAHC | BM |
| ENSG00000110693 | SOX6 | BM |
| ENSG00000105866 | SP4 | BM |
| ENSG00000189120 | SP6 | smoking duration |
| ENSG00000164266 | SPINK1 | smoking duration |
| ENSG00000133710 | SPINK5 | BM |
| ENSG00000152268 | SPON1 | BM |
| ENSG00000179954 | SSC5D | BM |
| ENSG00000136011 | STAB2 | BM |
| ENSG00000160828 | STAG3L2 | smoking duration |
| ENSG00000178078 | STAP2 | BM |
| ENSG00000159433 | STARD9 | BM |
| ENSG00000145087 | STXBP5L | smoking duration |
| ENSG00000159164 | SV2A | BM |
| ENSG00000147041 | SYTL5 | BM |
| ENSG00000163060 | TEKT4 | smoking duration |
| ENSG00000009694 | TENM1 | BM |
| ENSG00000270141 | TERC | BM |
| ENSG00000132604 | TERF2 | smoking duration |
| ENSG00000091513 | TF | smoking duration |
| ENSG00000087510 | TFAP2C | smoking duration |
| ENSG00000125780 | TGM3 | BM; smoking status |
| ENSG00000166948 | TGM6 | smoking status |
| ENSG00000163659 | TIPARP | smoking status |
| ENSG00000206432 | TMEM200C | smoking duration |
| ENSG00000214128 | TMEM213 | smoking duration |
| ENSG00000151715 | TMEM45B | smoking status |
| ENSG00000125247 | TMTC4 | smoking duration |
| ENSG00000185215 | TNFAIP2 | BM |
| ENSG00000143337 | TOR1AIP1 | BM |
| ENSG00000175274 | TP53I11 | smoking duration |
| ENSG00000131653 | TRAF7 | BM |
| ENSG00000072657 | TRHDE | smoking status |
| ENSG00000180098 | TRNAU1AP | smoking status |
| ENSG00000196428 | TSC22D2 | BM |
| ENSG00000104522 | TSTA3 | BM |
| ENSG00000156042 | TTC18 | BM |
| ENSG00000123607 | TTC21B | BM |
| ENSG00000155158 | TTC39B | smoking duration |
| ENSG00000213471 | TTLL13 | smoking duration |
| ENSG00000247596 | TWF2 | smoking duration |
| ENSG00000092445 | TYRO3 | smoking duration |
| ENSG00000137831 | UACA | BM |
| ENSG00000246922 | UBAP1L | smoking duration |
| ENSG00000154277 | UCHL1 | smoking status; smoking |
| | | duration |
| ENSG00000133958 | UNC79 | BM |
| ENSG00000006611 | USH1C | smoking status |
| ENSG00000166348 | USP54 | smoking status |
| ENSG00000114374 | USP9Y | gender |
| ENSG00000183878 | UTY | gender |
| ENSG00000162738 | VANGL2 | BM |
| ENSG00000160131 | VMA21 | BM |
| ENSG00000104142 | VPS18 | BM |
| ENSG00000095787 | WAC | BM |
| ENSG00000185798 | WDR53 | smoking duration |
| ENSG00000122574 | WIPF3 | smoking duration |
| ENSG00000070540 | WIPI1 | BM |
| ENSG00000126562 | WNK4 | BM |
| ENSG00000114251 | WNT5A | smoking status |
| ENSG00000180667 | YOD1 | BM |
| ENSG00000169155 | ZBTB43 | BM |
| ENSG00000198939 | ZFP2 | smoking duration |
| ENSG00000196867 | ZFP28 | smoking duration |
| ENSG00000106261 | ZKSCAN1 | smoking status |
| ENSG00000167840 | ZNF232 | smoking duration |
| ENSG00000188994 | ZNF292 | BM |
| ENSG00000124613 | ZNF391 | smoking duration |
| ENSG00000198795 | ZNF521 | BM |
| ENSG00000124444 | ZNF576 | smoking duration |
| ENSG00000258405 | ZNF578 | BM |
| ENSG00000197566 | ZNF624 | smoking duration |
| ENSG00000019995 | ZRANB1 | BM |

### Data Analysis

Samples may be classified using a trained classifier algorithm. Illustrative algorithms include but may not be limited to methods that reduce the number of variables such as principal component analysis algorithms, partial least squares methods, and independent component analysis algorithms. Illustrative algorithms further include but may not be limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, linear regression algorithms, and regularized linear discriminant analysis. Machine learning techniques include bagging procedures, boosting procedures, random forest algorithms, and combinations thereof. Cancer Inform, 2008; 6:77-97 provides an overview of the classification techniques provided above for the analysis of microarray intensity data.

The subject methods and algorithms enable: 1) gene expression analysis of samples containing low amount and/or low quality of nucleic acid; 2) a significant reduction of false positives and false negatives, 3) a determination of the underlying genetic, metabolic, or signaling pathways responsible for the resulting pathology, 4) the ability to assign a statistical probability to the accuracy of a diagnosis, a risk of developing a condition, a monitoring of changes in a condition, an effectiveness of an interventive therapy, or combinations thereof, 5) the ability to resolve ambiguous results, and 6) the ability to distinguish between lung conditions or sub-types of lung conditions.

The present disclosure provides for upfront methods of determining the cellular make-up of a particular biological sample so that the resulting molecular profiling signatures may be calibrated against the dilution effect due to the presence of other cell and/or tissue types. This upfront method may be an algorithm that uses a combination of cell and/or tissue specific gene expression patterns as an upfront mini-classifier for one or more or each component of the sample. This algorithm may use the gene expression patterns, or molecular fingerprint, to pre-classify the samples according to their composition and then apply a correction/normalization factor. Then, this data may feed in to an additional classification algorithm which may incorporate that information to aid in a further determination that a sample may be benign or malignant.

Raw gene expression level and alternative splicing data may be improved through the application of algorithms designed to normalize and or improve the reliability of the data. Data analysis may require a computer or other device, machine or apparatus for application of the various algorithms described herein due to the large number of individual data points that may be processed.

In some cases, the robust multi-array Average (RMA) method may be used to normalize the raw data. The RMA method begins by computing background-corrected intensities for each matched cell on a number of microarrays. The background corrected values may be restricted to positive values as described by Irizarry et al. Biostatistics 2003 Apr. 4 (2):249-64, which is entirely incorporated herein by reference. After background correction, the base-2 logarithm of each background corrected matched-cell intensity may be then obtained. The background corrected, log-transformed, matched intensity on each microarray may be then normalized using the quantile normalization method in which for each input array and each probe expression value, the array percentile probe value may be replaced with the average of all array percentile points, this method may be more completely described by Bolstad et al. Bioinformatics 2003, which is entirely incorporated herein by reference. Following quantile normalization, the normalized data may then be fit to a linear model to obtain an expression measure for each probe on each microarray. Tukey's median polish algorithm (Tukey, J. W., Exploratory Data Analysis. 1977), which is entirely incorporated herein by reference, may then be used to determine the log-scale expression level for the normalized probe set data.

Data may further be filtered to remove data that may be considered suspect. In some embodiments, data deriving from microarray probes that have fewer than about: 1, 2, 3, 4, 5, 6, 7 or 8 guanosine+cytosine nucleotides may be considered to be unreliable due to their aberrant hybridization propensity or secondary structure issues. A microarray probe having more than about 4 guanosine+cytosine nucleotides may be considered unreliable. A microarray probe having more than about 6 guanosine+cytosine nucleotides may be considered unreliable. A microarray probe having more than about 8 guanosine+cytosine nucleotides may be considered unreliable. A microarray probe having from about 4 guanosine+cytosine nucleotides to about 8 guanosine+cytosine nucleotides may be considered unreliable. Similarly, data deriving from microarray probes that have more than about: 8, 9, 10, 11, 12, 13, 14, 15, 16, 17, 18, 19, 20, 21, 22, 23, 24, 25 guanosine+cytosine nucleotides may be considered unreliable due to their aberrant hybridization propensity or secondary structure issues. A microarray probe having more than about 10 guanosine+cytosine nucleotides may be unreliable. A microarray probe having more than about 15 guanosine+cytosine nucleotides may be unreliable. A microarray probe having more than about 20 guanosine+cytosine nucleotides may be unreliable. A microarray probe having more than about 25 guanosine+cytosine nucleotides may be unreliable. A microarray probe having from about 8 guanosine+cytosine nucleotides to about 30 guanosine+cytosine nucleotides may be unreliable. A microarray probe having from about 10 guanosine+cytosine nucleotides to about 30 guanosine+cytosine nucleotides may be unreliable. A microarray probe having from about 12 guanosine+cytosine nucleotides to about 30 guanosine+cytosine nucleotides may be unreliable. A microarray probe having from about 15 guanosine+cytosine nucleotides to about 30 guanosine+cytosine nucleotides may be unreliable.

In some cases, unreliable probe sets may be selected for exclusion from data analysis by ranking probe-set reliability against a series of reference datasets. For example, RefSeq or Ensembl (EMBL) may be considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion. In some cases, the Ensembl high throughput cDNA and/or mRNA reference datasets may be used to determine the probe-set reliability separately or together. In other cases, probe-set reliability may be ranked. For example, probes and/or probe-sets that match perfectly to all reference datasets may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there may be many methods understood in the art for assessing the reliability of a given probe and/or probe-set for molecular profiling and the methods of the present disclosure encompass any of these methods and combinations thereof.

Methods of data analysis of gene expression levels or of alternative splicing may further include the use of a feature selection classifier algorithm as provided herein. In some embodiments of the present disclosure, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420), which is entirely incorporated herein by reference.

Methods of data analysis of gene expression levels and or of alternative splicing may further include the use of a pre-classifier algorithm. For example, an algorithm may use a cell-specific molecular fingerprint to pre-classify the samples according to their genetic composition, such as the expression of genes found within a cell (e.g., RNA found in a basal cell or RNA found in a blood cell) and then apply a correction/normalization factor. This data/information may then be fed in to a final classification algorithm which may incorporate that information to aid in a final classification, diagnosis or prognosis, or monitoring evaluation.

Methods of data analysis of gene expression levels and or of alternative splicing may further include the use of a classifier algorithm as provided herein. In some embodiments of the present disclosure a support vector machine (SVM) algorithm, a random forest algorithm, or a combination thereof is provided for classification of microarray data. In some embodiments, identified markers that distinguish samples (e.g., benign vs. malignant, normal vs. malignant, low risk vs. high risk) or distinguish types (e.g., ILD vs. lung cancer) may selected based on statistical significance. In some cases, the statistical significance selection is performed after applying a Benjamini Hochberg correction for false discovery rate (FDR).

Methods of data analysis of gene expression levels may further include the use of a principal component analysis (PCA). Principal component analysis can comprise a mathematical algorithm to reduce the dimensionality of data while retaining variation of the data set. The reduction can be accomplished by identifying principal components that correspond to maximal variations in the data. (See, e.g., Ringner et al, Nature Biotechnology, Vol. 26, No. 3, Mar. 2008). These principal components are described herein as Principal Components (PC) such as Cell type PC 1, Cell type PC 2, Cell type PC 3, batch PC 1, batch PC 2, and batch PC 3.

### Computer Systems

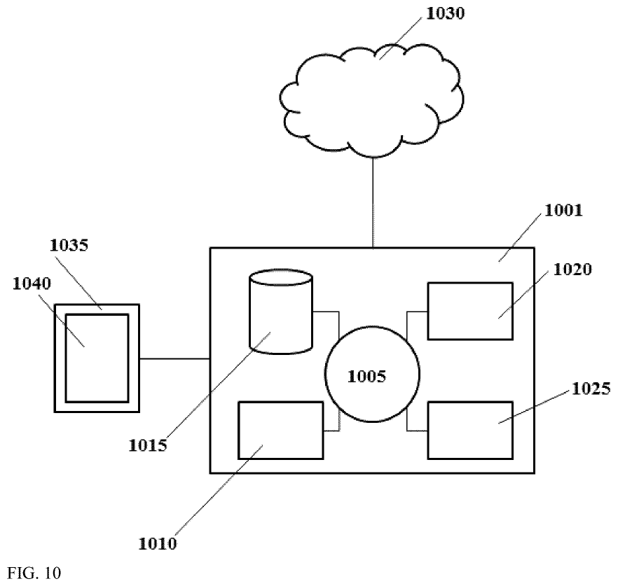

The present disclosure provides computer systems for implementing methods provided herein. FIG. 10 shows an example of a computer system 1001. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an interne and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., remote cloud server). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005.

### Treatments

Treatment may be provided or administered to a subject based on a classification of subject's sample as positive or negative for a condition, such as lung cancer. A treatment may be an intervention by a medical professional or in the form of providing actionable information to a subject in the form a tangible report (e.g., delivered through a computer system to be displayed to a subject on a graphical user interface, or a paper copy of a report).

An intervention by a medical profession may involve, by way of non-limiting examples, screening, monitoring, or administering therapy. Screening may include various imaging, or diagnostic testing techniques. Screening using imaging may include a CT scan, a low-dose computerized tomography (CT) scan, MM, and X-ray. In a non-limiting example, methods and systems of the present disclosure may be used after a lung nodule is identified in an imaging scan. Imaging may be used to screen or monitor a subject after he or she receives classification results. Diagnostic assays may similarly be used to identify a subject as a candidate for use of the methods of systems disclosed in the instant application. Such assays may include but are not limited to sputum cytology, tissue sample biopsy, immunoblot analysis, RNA sequencing or genome sequencing. Monitoring may involve a low-dose computerized tomography (CT) scan, X-ray, sputum cytology, RNA sequencing or genome sequencing.

In the event that a lung condition, such as cancer, is detected using the systems and methods of the instant disclosure, a therapy may be administered to a subject in need thereof. A therapy may involve, for example, the administration of one or more therapeutic agents or a surgical procedure. Non-limiting examples of therapeutic agents include chemotherapeutic agents, monoclonal antibodies, antibody drug conjugates, EGFR inhibitors, and ALK protein binding agents. A surgical procedure may involve, but is not limited to, thoracotomy, lobectomy, thoracoscopy, segmentectomy, wedge resection, or pneumonectomy . Treatment or therapy may include but is not limited to chemotherapy, radiation therapy, immunotherapy, hormone therapy, and pulmonary rehabilitation.

A treatment may be a medical intervention in the form of a report provided to a subject or to a medical professional. A medical professional may act as an intermediary and deliver results directly to a subject. The report may provide information such as the presence or absence of gene fusion(s) and results generated from classifying a sample as positive or negative for a lung condition based in part on assaying nucleic acids from epithelial cells in the subject's respiratory tract, such as lung cancer. The report may provide information regarding potential treatment options, such as potential drugs or clinical trials, based in part on the fusions detected.

By way of illustrative example, if a sample is classified as positive for lung cancer using the systems or methods of the present disclosure, then the subject may receive one or more of chemotherapy, radiation therapy, immunotherapy, hormone therapy, pulmonary rehabilitation, or any combination thereof. In another non-limiting example, if a sample is classified as negative for lung cancer using the systems or methods of the present disclosure, then the subject may be monitored on an on-going basis for potential development of cancerous nodules or lesions.

### EXAMPLES

### Example 1—Blood Index and Exclusion Criteria

The collection of nasal brushings (nasal swabs) may cause bleeding and result in blood contamination in the collected nasal brushing samples. It was theorized that blood contamination could impact classification scores. A blood index was developed to eliminate a substantial impact from blood that could alter the classifier performance. The blood index can be used to estimate a blood content within a sample. Samples with greater than 50% blood contamination can be excluded.

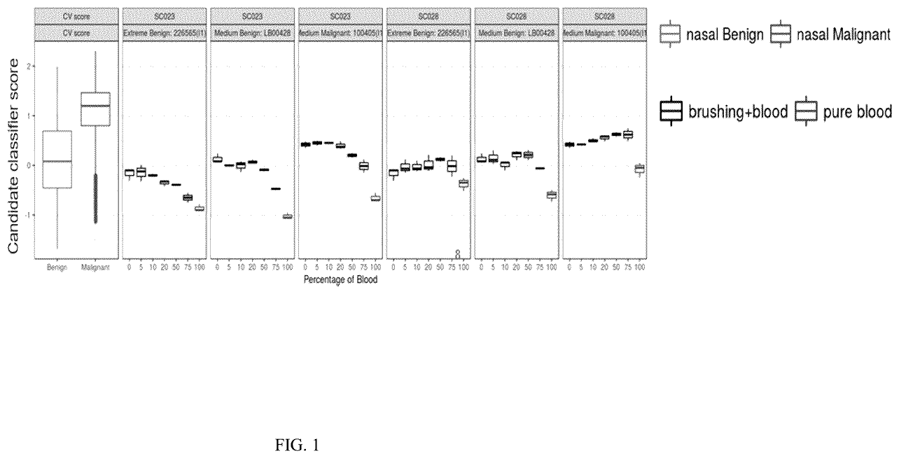

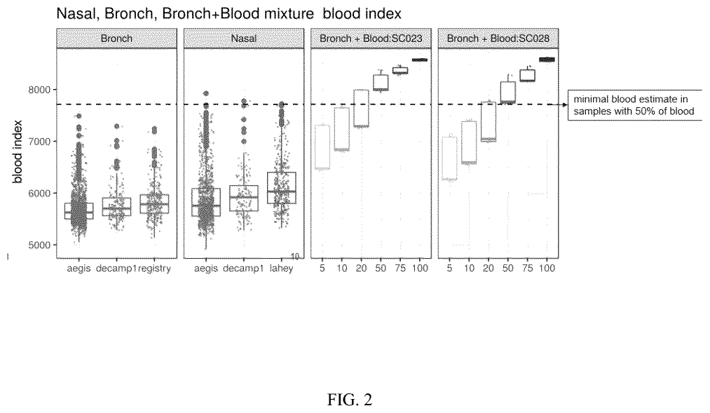

As can be seen in FIG. 1, pure blood scores low in nasal classifier (i.e. in the low-risk region); thus severe blood contamination may have an effect of pulling a nasal sample's score down only when blood contamination is severe (e.g. >50%). The blood index can be used to measure the level of blood in nasal samples. As can be seen in FIG. 2, a blood index >7713 is equivalent to a blood contamination of >50%. Approximately 0.2% of samples tested had this level of blood contamination.

### Example 2—Normalization Using RNA Yield and Library Diversity

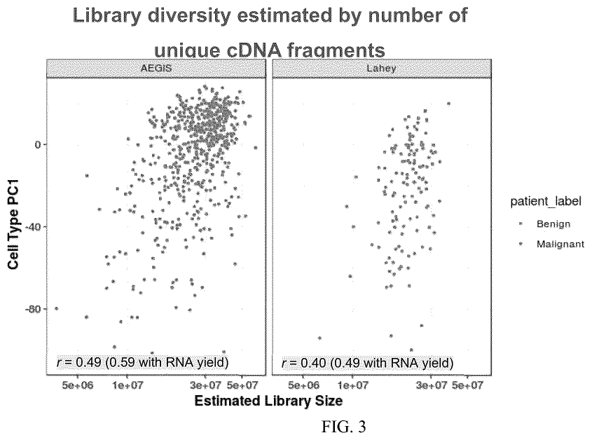

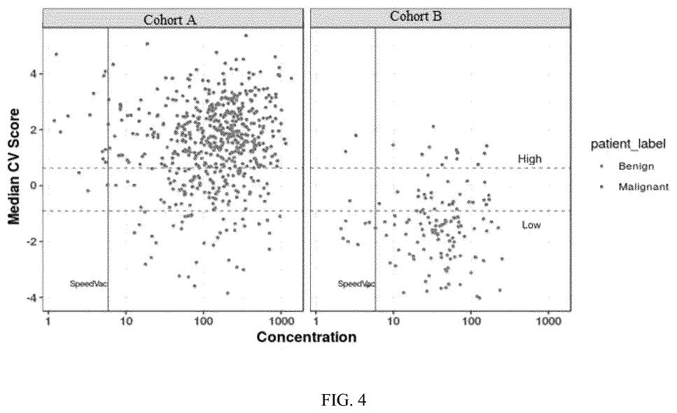

It was observed that RNA yield was correlated with genomic expression variability. A standardized RNA input was used in the UA assay to generate a comparable and stable genomic expression profile. The RNA yield concentration in training samples ranges from 1 ng/μL to greater than 1300 ng/μL. Samples with less than 5.88 ng/μL concentration need to be concentrated to 5.88 ng/μL prior to normalization. As can be seen in FIG. 3, library size is correlated with cell type PC1. As can be seen in FIG. 4, low RNA yield (less than 5.88 ng/μL) had no impact on classifier performance.

### Example 3—Controlling for UA Technical Variability

Variability can be defined as a fluctuation in gene expression. It could be a signal of interest (i.e., related to benign or malignant samples), or be noise. Noise is a type of variability that is not directly linked to a risk of sample being associated a risk of lung cancer. Variability and noise can come from may different sources along a sample process. In order to isolate and evaluate contributions from individual sources to separate noise from a risk of malignancy signal, the algorithm was tested for biological variability and technical variability (before and after sequencing). Biological variability includes smoking status and known lung conditions (such as asthma). Technical variability before sequencing includes brushing collection, blood contamination, storage and shipping, and RNA extraction. Technical variability during sequencing includes library preparation, exome capture, sequencing batches, and variability between research sample processing and CLIA regulated sample processing.

Technical variability in sequencing can be directly measured by technical replicates of samples run multiple times. Technical replicates of five nasal brushing samples (“sentinels”) were included in each 96-well plate run. A small set of genes with a large technical variability were identified based on the top 5 PCs. The PCA was repeated and 300 genes with a large contribution to the top 3 PCs were identified. The top 3 PCs were then recalculated using the 300 genes previously identified, and batch PC1 genes were regressed out from the expression data from all samples to normalize expression data for the identified technical variability. This was repeated for five cell-types: PC1, PC2, PC3, PC4 and PC5. 909 genes with high weights in the top 5 PCs were then excluded from downstream analyses.

### Example 4—Regressing Out Batch PC1 (rb1) Normalization to Control Technical Variability During Sequencing

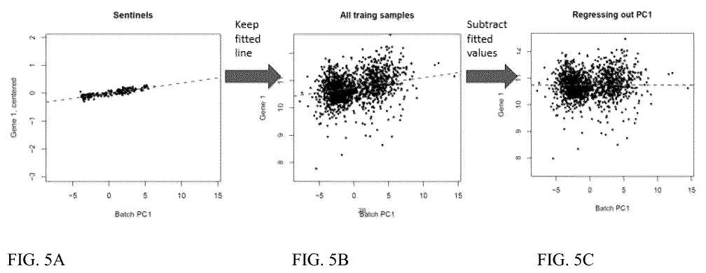

As can be seen in FIG. 5, the effect of batch PC1 was removed from expression data using regression-based adjustment. A regression line was calculated using centered expression from sentinels for each gene. The effect of batch PC1 was removed from the expression data of all samples using estimated regression lines.

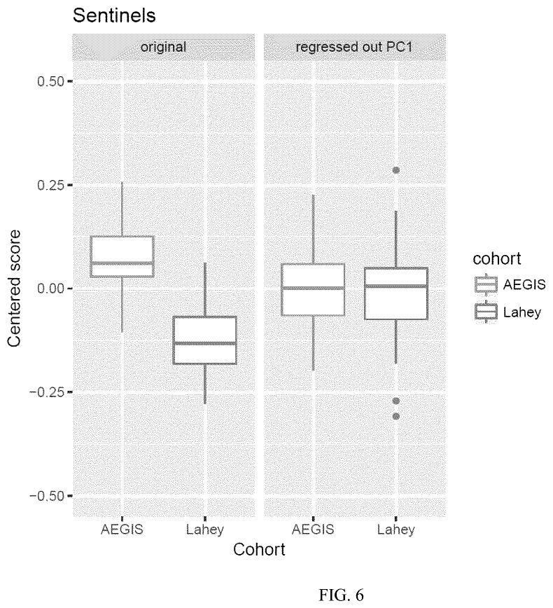

The normalization was tested on nasal brushing samples from individuals in the Cohort A and Cohort B databases. Rb1 normalization reduced technical variability by 10%. As can be seen in FIG. 6, regression of PC1 genes resulted in a normalization of scores for samples from both the Cohort A and Cohort B databases.

### Example 5—Regressing Out Normalization to Control Technical Variability Before Sequencing

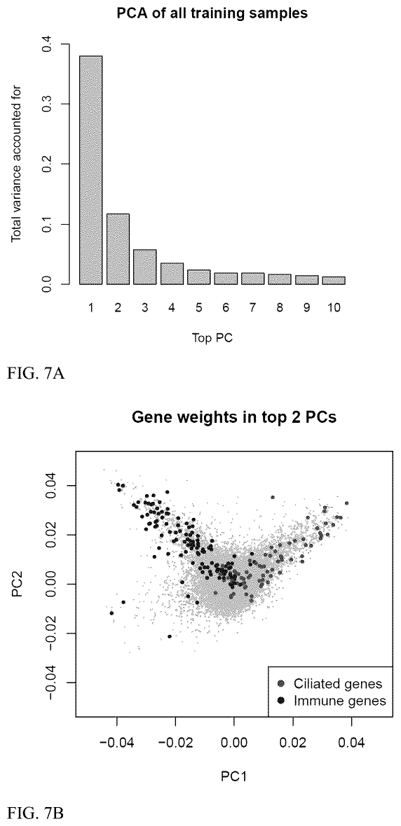

It can be difficult to isolate and control for individual contributing factors in biological variability and technical variability before sequencing at a gene expression level. It was found that current/former smoking status could be accounted for in the classifier, and the effect of blood contamination was small (see Example 1). To normalize for technical variability during sequencing, a PCA was run using all training samples. 300 genes with large contributions in the top PCs were identified. The top cell type PCs were recalculated using the 300 genes. Cell type PC1 or PC2 is then regressed out from the expression data of all samples. 930 primary training samples were tested. As can be seen in FIG. 7A, the top two PCs account for 50% of total variance. As can be seen in FIG. 7B, genes with high weights in the top two PCs contained many cell-type related genes, specifically ciliated genes and immune genes.

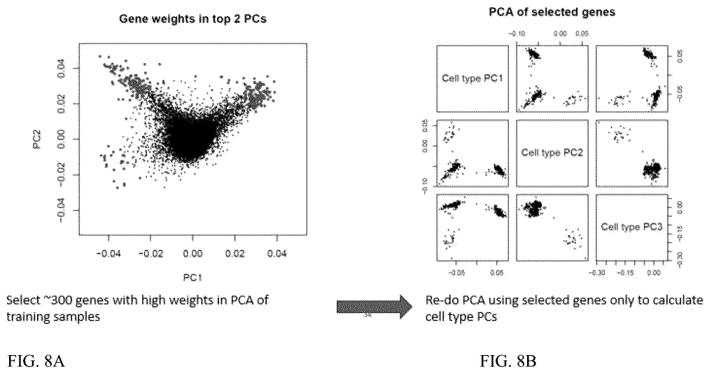

As can be seen in FIG. 8A and 8B, approximately 300 genes with the highest weights in the calculated PCA of training samples were selected and the PCA was re-run using the selected genes only to calculate cell type PCs.

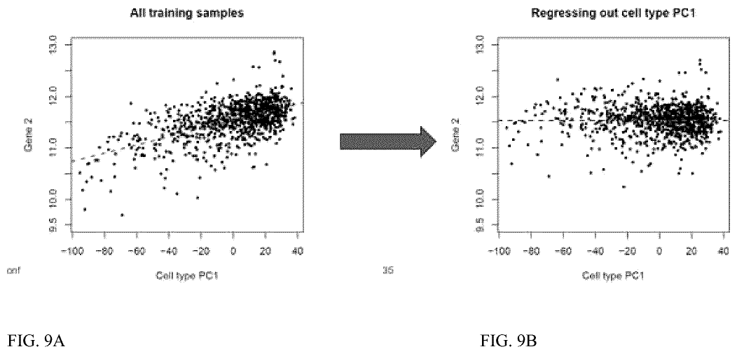

As can be seen in FIG. 9A cell type PCs were used as covariates in differential expression analysis to control for their effects on gene expression and included as candidate features in classifier training (FIG. 9A).

### Example 6: Regressing Out Batch PC1 and Cell Type PC1 and 2 (rb1rc12) Normalization and Including Cell Type PCs as Model Features

Cell type PCs and associated normalization were also used to control variability beyond UA sequencing. As can be seen in FIG. 9B, cell type PCs were regressed out of expression data similarly to batch PC1 in the normalization step.

### Example 7: Genomic Smoking Index

Smoking can result in acute and chronic gene expression changes. Over time, smoking can cause damage throughout the airway, known as the field of injury. Gene expression changes associated with this field of injury can aid with assessing a risk of a benign or malignant nodule. Smoking effect measured in the genomic space is both noise (a much stronger genomic signal that could potentially mask out a benign/malignant signal) and signal (when it results in genomic damage that is closely associated with benign/malignant signal). Developing smoking indexes can tease out the signal from the noise. A better benign/malignant signal separation was observed using a genomic smoking duration index as opposed to a clinical smoking years covariate.

### Genomic Smoking Status:

A genomic smoking status index (current versus former smoker) was developed comprising 80 genes.

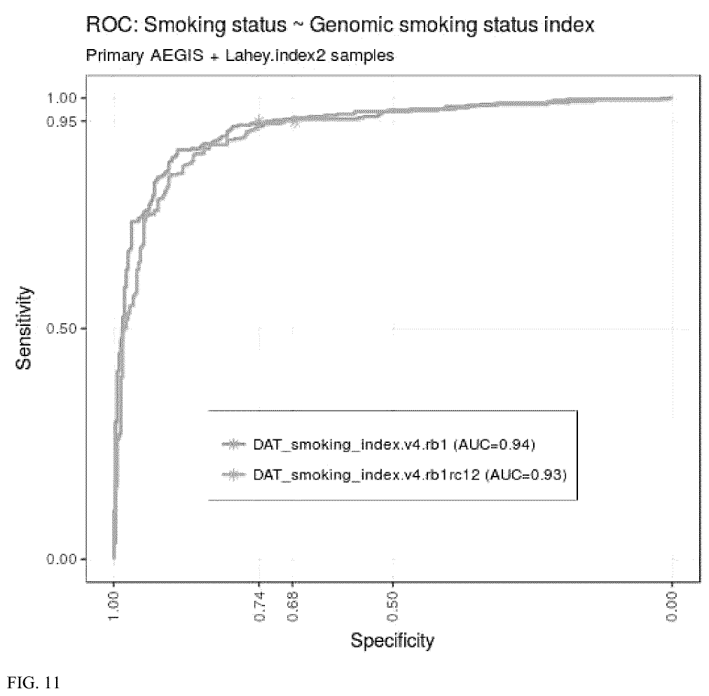

As can be seen in FIG. 11, the ROC of sensitivity versus specificity of a genomic smoking status index run on expression data subject to rb1 normalization or rb1rc12 normalization achieved excellent classification performance, with a very similar AUC (0.94 and 0.93, respectively) in a pool of 1,376 expression profiles pooled from the Cohort A, Cohort C1 and Cohort B databases.

### Genomic Smoking Duration:

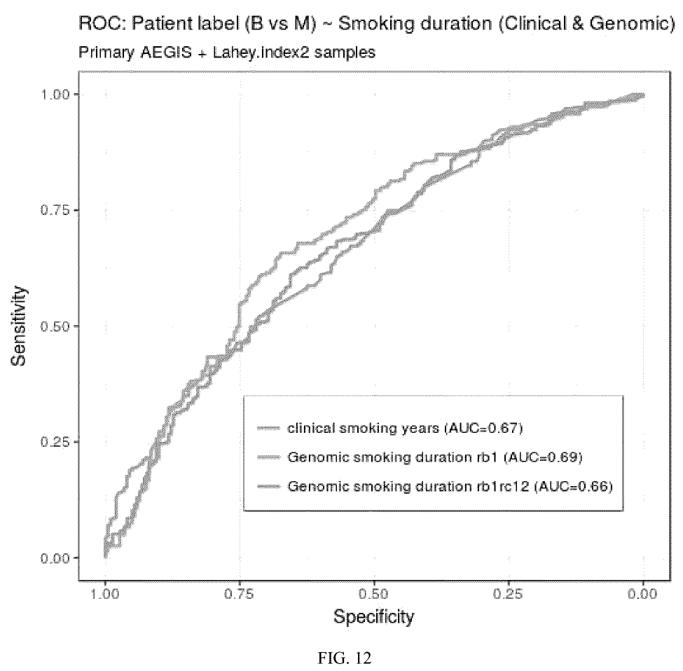

A smoking duration index was developed for each normalization protocol. For the rb1 normalization, a smoking duration of 193 genes was developed. For the rb1rc12 normalization, a smoking duration index of 187 genes was developed. As can be seen in FIG. 12, the smoking duration indexes showed a benign/malignant separation that was comparable or better than using a clinical smoking year covariate, indicating that an additional signal of malignancy had been captured using the smoking duration index. The AUC achieved using clinical smoking years was 0.67. The AUC achieved using the smoking duration index developed for the rb1 normalization was 0.69. The AUC achieved using the smoking duration index developed for the rb1rc12 normalization was 0.66.

### Example 8—Genomic Gender Index

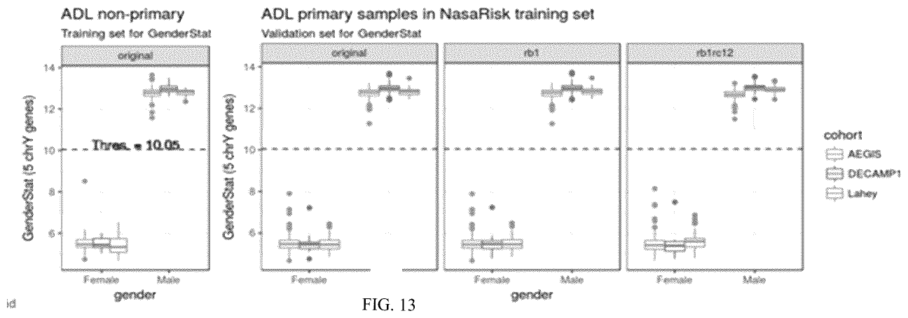

The expression levels of five chromosome Y genes were used to set a threshold value for biological sex of an individual to normalize gene expression. As can be seen in FIG. 13, between all databases (Cohort A, Cohort C1 and Cohort B) if the threshold value is greater than 10.05, the subject is identified as male. A 100% agreement with clinical gender was seen for both rb1 and rb1rc12 normalized gene expression data.

### Example 9—Defining Decision Boundaries

For each decision boundary, two definitions were considered. First, using the full model on the whole training set was considered to represent the true score-range. In order to avoid overfitting, a conservative buffer was built to mitigate the risk. Second, cross validated scores were averaged across 10 repeat samples to minimize overfitting and performance noise due to random variability. The score ranges of each of the two definitions may be different, therefore cut-offs were defined by both approaches in further validation studies.

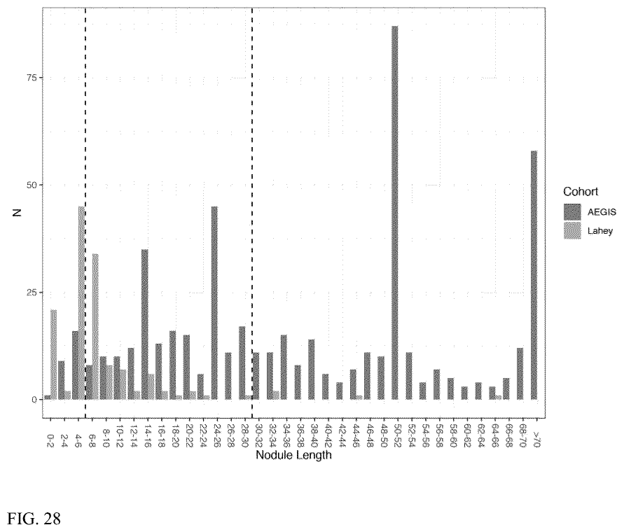

It was found that malignant samples from the Cohort B database scored slightly lower than malignant samples from the Cohort A database, even after rb1 and rb1rc12 normalization. For low-risk classifications, additional measures were implemented to ensure performance with the Cohort B database. As can be seen in FIG. 28, the length of nodules from the Cohort A subset are on average longer than the average nodule length of nodules from the Cohort B subset.

TABLE 2
Cohort B versus Cohort A Nodule Size

| Nodule Size | Cohort B | Cohort A | Combined |
|---|---|---|---|
| 6-30 mm | 64 (24%) | 198 (76%) | 262 |
| <=30 mm | 132 (37%) | 224 (63%) | 356 |
| No restriction | 137 (19%) | 580 (81%) | 717 |

TABLE 3
Overall prevalence of benign and
malignant nodules less than 6 mm

| Nodules <= 6 mm | Benign | Malignant |
|---|---|---|
| Cohort B | 63 (93%) | 5 (7%) |
| Cohort A | 16 (62%) | 10 (38%) |

Making a cutoff of less than or equal to 30 mm could maintain most of the Cohort B samples and reduce imbalances between the databases. It was found that for patients with nodules less than 6 mm, 90% were correctly called low risk. The remaining 10% were intermediate risk. Among truly malignant patients, ˜50% of them were classified as intermediate risk, providing them a critical opportunity for further assessment to catch the cancer early. The remaining 50% were called low risk. The performance between Cohort A and Cohort B in patients with nodules less than 6 mm were similar.

### Example 10: Comparison of Layered Structure versus Single Structure classifiers

TABLE 4
Overview of candidate classifiers

| Model | | | | | |
|---|---|---|---|---|---|
| Structure | Model | Normalization | Reason to include | concerns | Tier |
| Layered | A | rb1 | minimize cohort shift, ensure Lahey | >800 genes | 3 |
| | | | performance | | |
| | B | rb1rc12 | <800 genes, minimize cohort shift, | | 1 |
| | | | ensure Lahey performance | | |
| | C | rb1rc12 | <800 genes, minimize cohort shift, | ~3% lower specificity in low-risk | 2 |
| | | | no clinical pack-year | performance | |
| | D | rb1 | different model structure | ~7% lower specificity in low-risk | 3 |
| | | | (ensemble), no clinical pack-year | performance, >800 genes | |
| Single | E | rb1 | Best overall performance | cohort score shift, >800 genes | 3 |
| | F | rb1rc12 | <800 genes, no clinical smoking | moderate cohort score shift | 2 |
| | | | variables, high overall performance | | |

TABLE 5
Overview of candidate classifier performance

| | | Low-risk classification | | | | High-risk classification | | | |
|---|---|---|---|---|---|---|---|---|---|
| | | | | at 25% cancer | | | | at 25% cancer | |
| | | | | prevalence | | | | prevalence | |
| | | | | % classified | | | | % classified | |
| Model | AUC | Sensitivity | Specificity | as low-risk | NPV | Sensitivity | Specificity | as high-risk | PPV |
| A | 86/79 | 96 | 49 | 38% | 97% | 63 | 90 | 24% | 67% |
| B | 86/78 | 95 | 50 | 39% | 96% | 62 | 90 | 23% | 67% |
| C | 86/78 | 95 | 46 | 36% | 97% | 63 | 90 | 24% | 67% |
| D | 86/79 | 96 | 43 | 33% | 97% | 62 | 90 | 23% | 67% |
| E | 86 | 95 | 51 | 40% | 97% | 60 | 90 | 22% | 67% |
| F | 85 | 95 | 51 | 39% | 97% | 61 | 90 | 23% | 67% |

### Two-Layered Classification (Models A, B, C, and D)

To further refine the classification of samples with different risk profiles, a “top layer” classifier was developed to classify high risk samples. It was observed that clinical-heavy models identified high risk samples well. Top layer models were designed to comprise both genomic and clinical features, but clinical features were more highly weighted. A “bottom layer” model was also developed to score the remaining samples.

### Up-Stream Classifiers

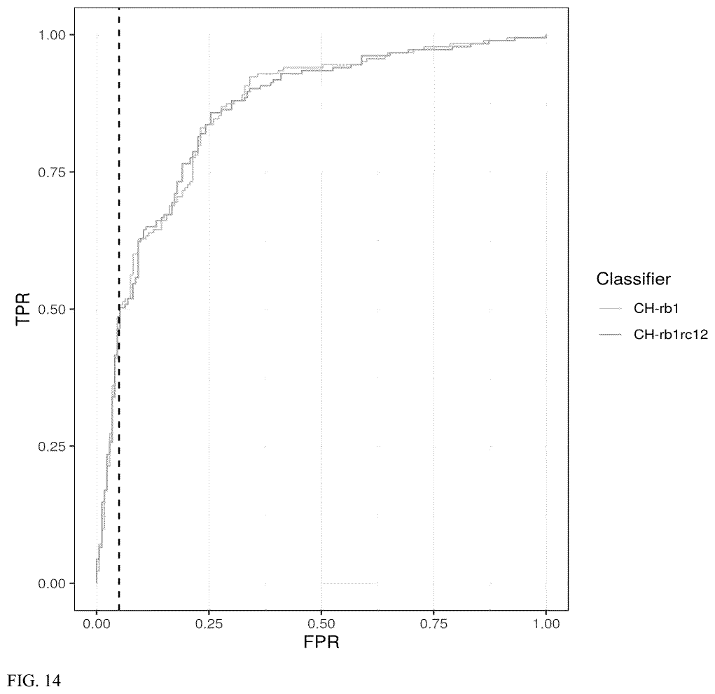

Both the top layer classifier and bottom layer classifier were trained on Cohort A, Cohort C and Cohort B cohorts. A linear regression model comprising clinical variables of age, Log2 nodule length, years since quit, speculation, and smoking duration index were used. As can be seen in FIG. 14, the classifier was run with both rb1 normalization and rb1rc12 normalization and the smoking duration index. As described previously, rb1 normalization with the smoking duration index measured 193 genes and rb1rc12 normalization with the smoking duration index measured 187 genes.

The results are summarized below.

TABLE 6
Clinical Heavy Upstream Classifier Performance

| Clinical | | | | | | |
|---|---|---|---|---|---|---|
| heavy | | Sensitivity@ | Number | Prevalence | Number remain | Prevalence in |
| upstream | | Specificity | classified | in high risk | intermediate | intermediate |
| classifier | AUC | 95% | as high risk | samples | risk | samples |
| CH-rb1 | 0.86 | 50% | 101 (28.4%) | 91.1% | 255 (71.6%) | 35.7% |
| CH-rb1rc12 | 0.86 | 49% | 100 (28.1%) | 91.0% | 256 (71.9%) | 36.1% |

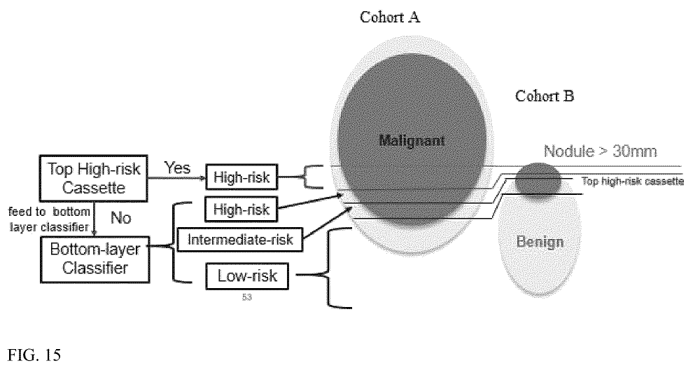

As can be seen in FIG. 15, if a sample is not identified as high risk by the top layer (“top high-risk cassette”) it is fed to the bottom layer classifier. A representation of overlap in nodule size between the Cohort A and Cohort B subsets is shown in the circles under each identifier “Cohort A” and “Cohort B”, wherein the dark circle represents a proportion of malignant samples and the light circles represent a proportion of benign samples in each database.

TABLE 7
Two-Layer Classifier Performance:

| Cohort A and | | | | |
|---|---|---|---|---|
| Cohort B, | | | | |
| Nodules <= | | N Samples | N Cohort A | N Cohort B |
| 30 mm | Action | (Prevalence) | (Prevalence) | (Prevalence) |
| | | 356 (51.4%) | 224 (69.6%) | 132 (20%) |
| CH-rb1 | Classified as | 101 (91.1%) | 95 (91.6%) | 6 (83.3%) |
| | high risk | | | |
| | Intermediate | 255 (35.7%) | 129 (53.3%) | 126 (17.5%) |
| | risk to | | | |
| | bottom layer | | | |
| | classifier | | | |
| CH-rb1rc12 | Classified as | 100 (91.0%) | 94 (91.6%) | 6 (83.3%) |
| | high risk | | | |
| | Intermediate | 256 (36.1%) | 130 (54.0%) | 126 (17.5%) |
| | risk to | | | |
| | bottom layer | | | |
| | classifier | | | |

### Example 11: rb1 Normalization Layered Candidate Classifier Performance (Model A)

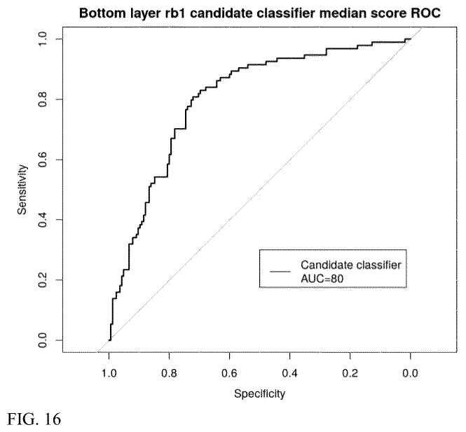

As can be seen in FIG. 16, the classifier performance achieved an AUC of 0.8 in an ROC analysis of sensitivity versus specificity. The model structure was a SVM model with covariate X gene and covariate X genomic index interaction, with hierarchical clustering of the top 20% of gene features. The features are summarized in the table below.

TABLE 8
Features of Model A Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 1029 | 1029 |
| Cohort C + | Cohort C + | status, rin, | length, nodule | Smk.idx.v4.rb1 | | |
| Cohort B | Cohort B | celltype PC1-3 | spiculation, | Smk.duration.idx.v0.rb1 | | |
| (idx2) | (idx2) | batch PC1-3 | piecewise pack | Batch PC2-3 | | |
| | | | year (<20, | Celltype PC1-3 | | |
| | | | 20-50, >50) | | | |
| | | | Up-stream | | | |
| | | | additional: | | | |
| | | | Years Since Quit | | | |

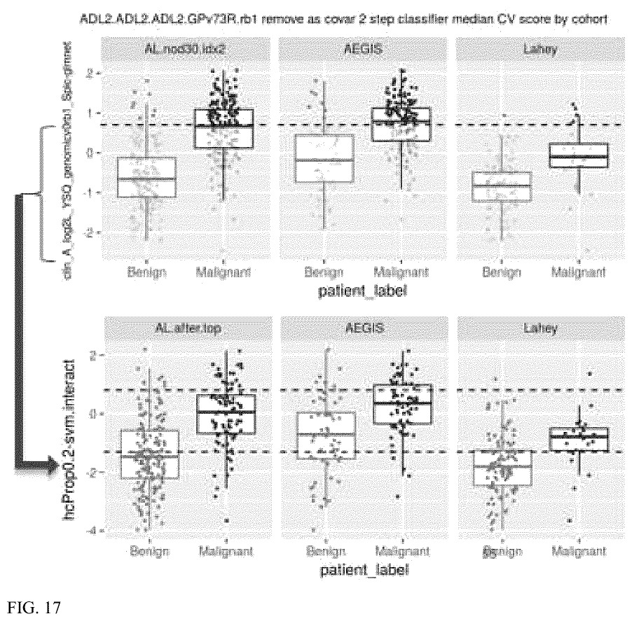

As can be seen in FIG. 17, the classification decision boundary for high-risk classification was well separated from benign samples in the top layer. The bottom layer classifier low-risk classification decision boundary was chosen to ensure sensitivity to samples from the Cohort B database. The results are summarized below:

TABLE 9
Model A performance, score by step

| | AUC | Classification | Sensitivity | Specificity |
|---|---|---|---|---|
| Top layer | 86 | High-risk | 50 | 95 |
| Bottom | 79 | Low-risk | 92 | 52 |
| Layer | | High-risk | 25 | 95 |

TABLE 10
Model A performance, overall score

| | Classi- | Sensi- | Speci- | | |
|---|---|---|---|---|---|
| Cohort | fication | tivity | ficity | @ 25% cancer prevalence | |
| Cohort A | Low- | 98 | 26 | % classified | NPV |
| | risk | | | as low-risk | |
| | | | | 20% | 97% |
| | High- | 70 | 76 | % classified | PPV |
| | risk | | | as high-risk | |
| | | | | 35% | 50% |
| Cohort B | Low- | 85 | 62 | % classified | NPV |
| | risk | | | as low-risk | |
| | | | | 50% | 93% |
| | High- | 22 | 98 | % classified | PPV |
| | risk | | | as high-risk | |
| | | | | 7% | 80% |

TABLE 11
Model A performance, combined median cross-validation
performance versus Benchmark Gould model performance

| Classifi- | Sensi- | Speci- | | |
|---|---|---|---|---|
| cation | tivity | ficity | Extrapolation @ 25% cancer prevalence | |
| Low-risk- | 96 | 49 | % classified | NPV |
| median | | | as low-risk | |
| cross- | | | 38% | 97% |
| validation | | | | |
| High-risk- | 63 | 90 | % classified | PPV |
| median | | | as high-risk | |
| cross- | | | 24% | 67% |
| validation | | | | |
| Low-risk- | 96 | 34 | % classified | NPV |
| Gould | | | as low-risk | |
| | | | 27% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 49% specificity when classifying a low-risk (15% higher than Gould). The candidate classifier showed 63% sensitivity when classifying high-risk (9% higher than Gould). In a population with 25% cancer prevalence, the model stratified 62% of patients to low or high risk, while Gould only moved 48% of patients.

### Example 12: Down-Stream rb1rc12 Candidate Classifier Performance (Model B)

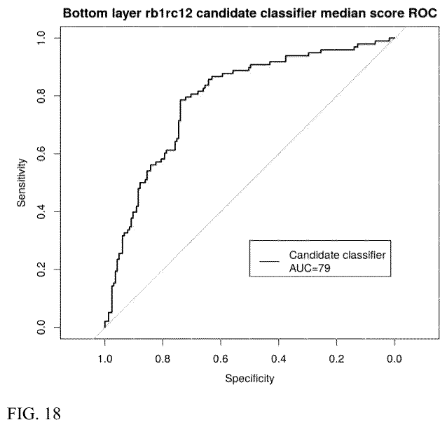

As can be seen in FIG. 18, the classifier performance achieved an AUC of 0.79 in an ROC gene and covariate X genomic index interaction, with HOPACH clustering of the top 20% of gene features. The features are summarized in the table below.

TABLE 12
Features of Model B Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 502 | 1083 |
| Cohort C + | Cohort B | status, rin, | length, nodule | Smk.idx.v4.rb1rc12 | | |
| Cohort B | (idx2) | celltype PC1-3 | spiculation, | Smk.duration.idx.v0.rb1rc12 | | |
| (idx2) | | batch PC1-3 | piecewise pack | | | |
| | | | year (<20, | | | |
| | | | 20-50, >50) | | | |
| | | | Up-stream | | | |
| | | | additional: | | | |
| | | | Years Since Quit | | | |

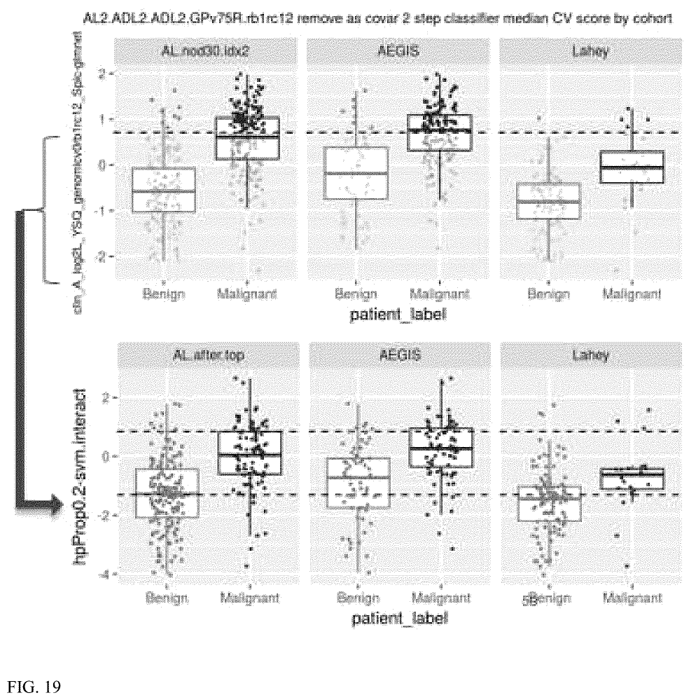

As can be seen in FIG. 19, the classification decision boundary for high-risk classification was well separated from benign samples in the top layer. The bottom layer classifier low-risk classification decision boundary was chosen to ensure sensitivity to samples from the Cohort B database. The results are summarized below:

TABLE 13
Model B performance, score by step

| | AUC | Classification | Sensitivity | Specificity |
|---|---|---|---|---|
| Top layer | 86 | High-risk | 49 | 95 |
| Bottom | 79 | Low-risk | 89 | 52 |
| Layer | | High-risk | 25 | 95 |

TABLE 14
Model B performance, overall score

| | Classifi- | Sensi- | Speci- | | |
|---|---|---|---|---|---|
| Cohort | cation | tivity | ficity | @ 25% cancer prevalence | |
| Cohort | Low-risk | 96 | 32 | % classified | NPV |
| A | | | | as low-risk | |
| | | | | 25% | 96% |
| | High-risk | 69 | 79 | % classified | PPV |
| | | | | as high-risk | |
| | | | | 32% | 53% |
| Cohort | Low-risk | 85 | 60 | % classified | NPV |
| B | | | | as low-risk | |
| | | | | 49% | 92% |
| | High-risk | 26 | 96 | % classified | PPV |
| | | | | as high-risk | |
| | | | | 9% | 69% |

TABLE 15
Model B performance, combined median cross-validation
performance versus Benchmark Gould model performance

| Classifi- | Sensi- | Speci- | | |
|---|---|---|---|---|
| cation | tivity | ficity | Extrapolation @ 25% cancer prevalence | |
| Low-risk- | 95 | 50 | % classified | NPV |
| median | | | as low-risk | |
| cross- | | | 39% | 96% |
| validation | | | | |
| High-risk- | 62 | 90 | % classified | PPV |
| median | | | as high-risk | |
| cross- | | | 23% | 67% |
| validation | | | | |
| Low-risk- | 95 | 44 | % classified | NPV |
| Gould | | | as low-risk | |
| | | | 34% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 50% specificity when classifying a low-risk (6% higher than Gould). The candidate classifier showed 62% sensitivity when classifying high-risk (8% higher than Gould). In a population with 25% cancer prevalence, the model stratified 62% of patients to low or high risk, while Gould only moved 55% of patients.

### Example 13: Down-Stream Few Clinvar Candidate Classifier Performance (Model C)

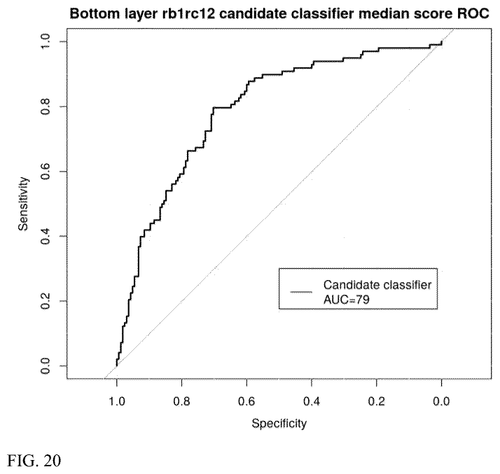

As can be seen in FIG. 20, the classifier performance achieved an AUC of 0.79 in an ROC gene and covariate X genomic index interaction, with HOPACH clustering of the top 50% of gene features. The features are summarized in the table below.

TABLE 16
Features of Model C Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 514 | 1099 |
| Cohort C + | Cohort B | status, rin, | length, nodule | Smk.idx.v4.rb1rc12 | | |
| Cohort B | (idx2) | celltype PC1-3 | spiculation, | Smk.duration.idx.v0.rb1rc12 | | |
| (idx2) | | batch PC1-3 | Up-stream | | | |
| | | | additional: | | | |
| | | | Years Since Quit | | | |

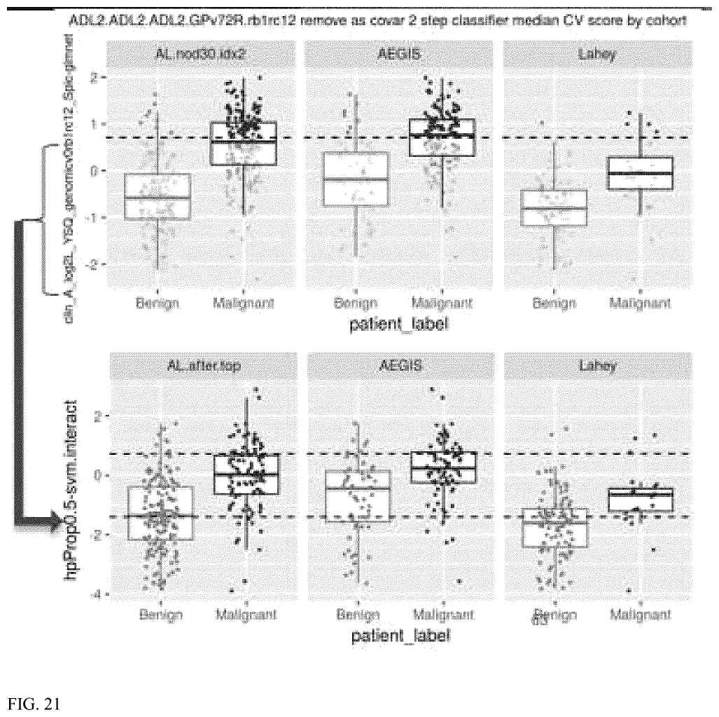

As can be seen in FIG. 21, the classification decision boundary for high-risk classification was well separated from benign samples in the top layer. The bottom layer classifier low-risk classification decision boundary was chosen to ensure sensitivity to samples from the Cohort B database. The results are summarized below:

TABLE 17
Model C performance, score by step

| | AUC | Classification | Sensitivity | Specificity |
|---|---|---|---|---|
| Top layer | 86 | High-risk | 49 | 95 |
| Bottom | 78 | Low-risk | 90 | 49 |
| Layer | | High-risk | 26 | 95 |

TABLE 18
Model C performance, overall score

| | Classifi- | Sensi- | Speci- | | |
|---|---|---|---|---|---|
| Cohort | cation | tivity | ficity | @ 25% cancer prevalence | |
| Cohort | Low-risk | 97 | 26 | % classified | NPV |
| A | | | | as low-risk | |
| | | | | 21% | 96% |
| | High-risk | 69 | 78 | % classified | PPV |
| | | | | as high-risk | |
| | | | | 34% | 51% |
| Cohort | Low-risk | 85 | 59 | % classified | NPV |
| B | | | | as low-risk | |
| | | | | 47% | 93% |
| | High-risk | 26 | 97 | % classified | PPV |
| | | | | as high-risk | |
| | | | | 9% | 75% |

TABLE 19
Model C performance, combined median cross-validation
performance versus Benchmark Gould model performance

| Classifi- | Sensi- | Speci- | | |
|---|---|---|---|---|
| cation | tivity | ficity | Extrapolation @ 25% cancer prevalence | |
| Low-risk- | 95 | 46 | % classified | NPV |
| median | | | as low-risk | |
| cross- | | | 36% | 97% |
| validation | | | | |
| High-risk- | 63 | 90 | % classified | PPV |
| median | | | as high-risk | |
| cross- | | | 24% | 67% |
| validation | | | | |
| Low-risk- | 95 | 44 | % classified | NPV |
| Gould | | | as low-risk | |
| | | | 34% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 46% specificity when classifying a low-risk (2% higher than Gould). The candidate classifier showed 63% sensitivity when classifying high-risk (9% higher than Gould). In a population with 25% cancer prevalence, the model stratified 60% of patients to low or high risk, while Gould only moved 55% of patients.

### Example 14: Down-Stream Ensemble Candidate Classifier Performance (Model D)

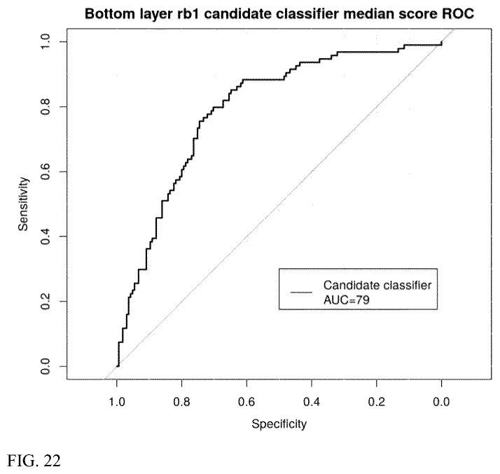

As can be seen in FIG. 22, the classifier performance achieved an AUC of 0.79 in an ROC gene and covariate X genomic index interaction, with hierarchical clustering of the top 10% of genes, HOPACH clustering of the top 10% of gene features, HOPACH clustering of the top 20% of gene features selected from all 3 cohorts and Cohort A and Cohort B only. The features are summarized in the table below.

TABLE 20
Features of Model D Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 1331 | 1331 |
| Cohort C + | Cohort B | status, rin, | length, nodule | Smk.idx.v4.rb1 | | |
| Cohort B | (idx2) | celltype PC1-3 | spiculation, | Smk.duration.idx.v0.rb1 | | |
| (idx2) | | batch PC1-3 | Up-stream | Batch PC2-3 | | |
| | | | additional: | Celltype PC1-3 | | |
| | | | Years Since Quit | | | |

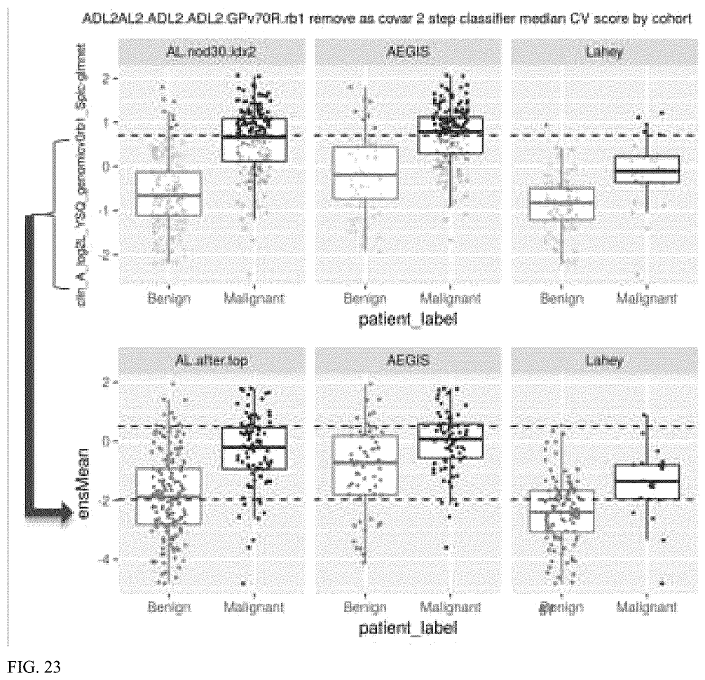

As can be seen in FIG. 23, the classification decision boundary for high-risk classification was well separated from benign samples in the top layer. The bottom layer classifier low-risk classification decision boundary was chosen to ensure sensitivity to samples from the Cohort B database. The results are summarized below:

TABLE 21
Model D performance, score by step

| | AUC | Classification | Sensitivity | Specificity |
|---|---|---|---|---|
| Top layer | 86 | High-risk | 50 | 95 |
| Bottom | 79 | Low-risk | 93 | 45 |
| Layer | | High-risk | 24 | 95 |

TABLE 22
Model D performance, overall score

| | Classi- | Sensi- | Speci- | | |
|---|---|---|---|---|---|
| Cohort | fication | tivity | ficity | @ 25% cancer prevalence | |
| Cohort | Low- | 98 | 18 | % classified | NPV |
| A | risk | | | as low-risk | |
| | | | | 33% | 97% |
| | High- | 69 | 76 | % classified | PPV |
| | risk | | | as high-risk | |
| | | | | 23% | 49% |
| Cohort | Low- | 85 | 58 | % classified | NPV |
| B | risk | | | as low-risk | |
| | | | | 49% | 92% |
| | High- | 22 | 98 | % classified | PPV |
| | risk | | | as high-risk | |
| | | | | 9% | 81% |

TABLE 23
Model D performance, combined median cross-validation
performance versus Benchmark Gould model performance

| Classi- | Sensi- | Speci- | | |
|---|---|---|---|---|
| fication | tivity | ficity | Extrapolation @ 25% cancer prevalence | |
| Low-risk- | 96 | 43 | % classified | NPV |
| median | | | as low-risk | |
| cross- | | | 33% | 97% |
| validation | | | | |
| High-risk- | 62 | 90 | % classified | PPV |
| median | | | as high-risk | |
| cross- | | | 23% | 67% |
| validation | | | | |
| Low-risk- | 96 | 34 | % classified | NPV |
| Gould | | | as low-risk | |
| | | | 27% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 43% specificity when classifying a low-risk (9% higher than Gould). The candidate classifier showed 62% sensitivity when classifying high-risk (8% higher than Gould). In a population with 25% cancer prevalence, the model stratified 56% of patients to low or high risk, while Gould only moved 48% of patients.

### Example 15: One-Step Classification Using the rb1 Candidate Classifier (Model E)

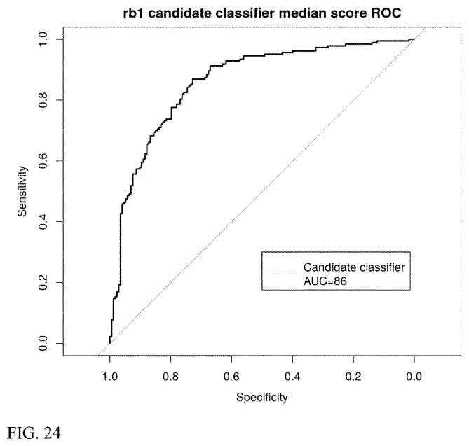

As can be seen in FIG. 24, the classifier performance achieved an AUC of 0.86 in an ROC gene and covariate X genomic index interaction, with HOPACH clustering of the top 20% of gene features. The features are summarized in the table below.

TABLE 24
Features of Model E Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 1092 | 1092 |
| Cohort C + | Cohort B | status, rin, | length, nodule | Smk.idx.v4.rb1 | | |
| Cohort B | (idx2) | celltype PC1-3 | spiculation, | Smk.duration.idx.v0.rb1 | | |
| (idx2) | | batch PC1-3 | piecewise pack | Batch PC2-3 | | |
| | | | year (<20, | Celltype PC1-3 | | |
| | | | 20-50, >50) | | | |

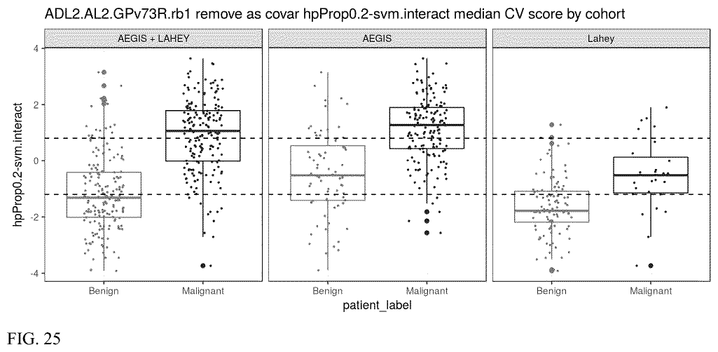

As can be seen in FIG. 25, the classification decision boundary for high-risk classification was well separated from benign samples. The results are summarized below:

TABLE 25
Model E performance

| | | | | | @ 25% cancer | |
|---|---|---|---|---|---|---|
| Cohort | AUC | Classification | Sensitivity | Specificity | prevalence | |
| Cohort A | 80 | Low-risk | 97 | 27 | % classified | NPV |
| | | | | | as low-risk | |
| | | | | | 21% | 97% |
| | | High-risk | 66 | 78 | % classified | PPV |
| | | | | | as high-risk | |
| | | | | | 33% | 50% |
| Cohort B | 77 | Low-risk | 78 | 66 | % classified | NPV |
| | | | | | as low-risk | |
| | | | | | 55% | 90% |
| | | High-risk | 20 | 98 | % classified | PPV |
| | | | | | as high-risk | |
| | | | | | 7% | 78% |

TABLE 26
Model E performance, combined median cross-validation
performance versus Benchmark Gould model performance

| Classi- | Sensi- | Speci- | | |
|---|---|---|---|---|
| fication | tivity | ficity | Extrapolation @ 25% cancer prevalence | |
| Low-risk- | 95 | 51 | % classified | NPV |
| median | | | as low-risk | |
| cross- | | | 40% | 97% |
| validation | | | | |
| High-risk- | 60 | 90 | % classified | PPV |
| median | | | as high-risk | |
| cross- | | | 22% | 67% |
| validation | | | | |
| Low-risk- | 95 | 44 | % classified | NPV |
| Gould | | | as low-risk | |
| | | | 34% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 51% specificity when classifying a low-risk (7% higher than Gould). The candidate classifier showed 60% sensitivity when classifying high-risk (6% higher than Gould). In a population with 25% cancer prevalence, the model stratified 62% of patients to low or high risk, while Gould only moved 55% of patients.

### Example 16: One-Step Classification Using the rb1rc12 Candidate Classifier (Model F)

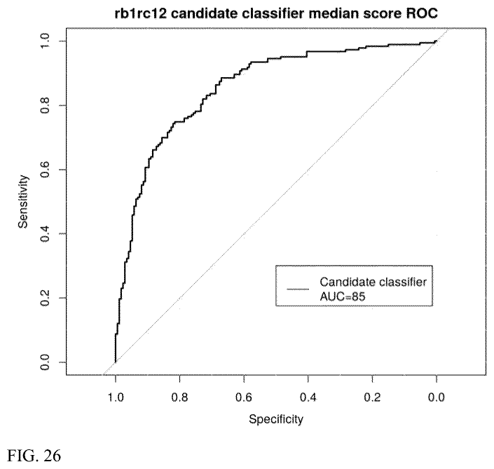

As can be seen in FIG. 26, the classifier performance achieved an AUC of 0.85 in an ROC analysis of sensitivity versus specificity. The model structure was a SVM model with covariate X gene and covariate X genomic index interaction, with hierarchical clustering of the top 10% of gene features. The features are summarized in the table below.

TABLE 27
Features of Model F Classifier

| | Differential | Differential | | | | # gene in |
|---|---|---|---|---|---|---|
| Training | Expression | Expression | Clinical | | # gene | model + |
| Set | Set | adjustment | Covariates | Genomic Index | in model | normalization |
| Cohort A + | Cohort A + | Gender, smoking | Age, log2 nodule | Gender | 747 | 1320 |
| Cohort C + | Cohort B | status, rin, | length, nodule | Smk.idx.v4.rb1rc12 | | |
| Cohort B | (idx2) | celltype PC1-3 | spiculation | Smk.duration.idx.v0.rb1rc12 | | |
| (idx2) | | batch PC1-3 | | | | |

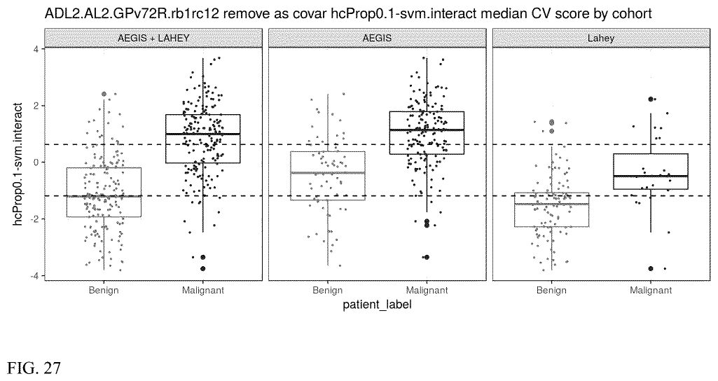

As can be seen in FIG. 27, the classification decision boundary for high-risk classification was well separated from benign samples in the top layer. The bottom layer classifier low-risk classification decision boundary was chosen to ensure sensitivity to samples from the Cohort B database. The results are summarized below:

TABLE 28
Model F performance

| | | | | | @ 25% cancer | |
|---|---|---|---|---|---|---|
| Cohort | AUC | Classification | Sensitivity | Specificity | prevalence | |
| Cohort A | 80 | Low-risk | 97 | 27 | % classified | NPV |
| | | | | | as low-risk | |
| | | | | | 21% | 96% |
| | | High-risk | 67 | 79 | % classified | PPV |
| | | | | | as high-risk | |
| | | | | | 32% | 52% |
| Cohort B | 78 | Low-risk | 81 | 65 | 0% classified | NPV |
| | | | | | as low-risk | |
| | | | | | 53% | 91% |
| | | High-risk | 26 | 97 | % classified | PPV |
| | | | | | as high-risk | |
| | | | | | 9% | 75% |

TABLE 29
Model F performance, combined median cross-validation
performance versus Benchmark Gould model performance

| Classi- | Sensi- | Speci- | | |
|---|---|---|---|---|
| fication | tivity | ficity | Extrapolation @ 25% cancer prevalence | |
| Low-risk- | 95 | 51 | % classified | NPV |
| median | | | as low-risk | |
| cross- | | | 39% | 97% |
| validation | | | | |
| High-risk- | 61 | 90 | % classified | PPV |
| median | | | as high-risk | |
| cross- | | | 23% | 67% |
| validation | | | | |
| Low-risk- | 95 | 44 | % classified | NPV |
| Gould | | | as low-risk | |
| | | | 34% | 96% |
| High-risk- | 54 | 90 | % classified | PPV |
| Gould | | | as high-risk | |
| | | | 21% | 65% |

The candidate two step classifier on the combined set achieved the user requirement in cross-validation evaluation. The candidate classifier showed 51% specificity when classifying a low-risk (7% higher than Gould). The candidate classifier showed 61% sensitivity when classifying high-risk (7% higher than Gould). In a population with 25% cancer prevalence, the model stratified 62% of patients to low or high risk, while Gould only moved 55% of patients.

### Example 17: Clinical-Genomic Classifier Development

Accurate assessment of risk of malignancy (ROM) is critical in patients with a screen-detected or incidental pulmonary nodule (PN). We sought to validate a clinical-genomic classifier utilizing RNA whole-transcriptome sequencing of cells from the nasal epithelium of individuals who have smoked with a PN.

A classifier utilizing genomic data from nasal brushings and clinical features was trained on a set of 1120 patients. Performance of the 502 gene classifier was validated in a set of 249 patients with results extrapolated to a population with 25% cancer prevalence. We measured performance in PN <8 mm and ≥8 mm and lung cancers by stages and histology. The cohort was expanded to include a set of patients with a history of non-lung cancer.

### Study Design

Study procedures, endpoints, analyses, and sub-analyses were pre-specified in a Design Control product development process. This study utilized nasal brushing samples from three cohorts of individuals with a solid, part-solid or ground glass PN: the Airway Epithelial Gene Expression in the Diagnosis of Lung Cancer (AEGIS-1 and AEGIS-2) cohorts, and the Lahey lung cancer screening cohort. Patients were followed until final diagnosis or for a at least 12 months. Nasal specimens were collected with a soft cytology brush lateral to the inferior turbinate. Institutional review board (IRB) approval was obtained by each participating institution prior to study commencement, and informed consent was obtained from all patients.

A total of 1744 evaluable patients (344 from Lahey and 1400 from AEGIS-1 and 2) with a suspicious lung lesion were allocated for the development and validation of the nasal swab classifier through randomization: 1120 (211 from Lahey and 909 from AEGIS-1 and 2) were allocated to training and 624 (133 from Lahey and 491 from AEGIS) to validation. Subjects were further excluded from the primary validation set due to prior or concurrent cancer (138 pts), missing nodule size, nodule size >30 mm or for samples that did not meet acceptable shipping criteria (237 patients. This resulted in a primary validation set of 249 patients (90 from Lahey and 159 from AEGIS-1 and 2). A diagnosis of lung cancer was established by cytology or pathology, or in circumstances where a presumptive diagnosis of cancer led to definitive ablative therapy without pathology. Patients who were defined as benign had a specific diagnosis of a benign condition or radiographic stability or resolution at ≥12 months.

### Sample Collection, RNA Extraction, Amplification, and Sequencing

Nasal specimens utilized for classifier training and validation were collected using a Cytopak Cyto-Soft brush (CP-5B). After sample collection, nasal brush specimens were stored in a nucleic acid preservative (RNAprotect, QIAGEN, Hilden, Germany) and either shipped chilled to a contract research lab for RNA extraction (AEGIS) or frozen at −80° C. prior to RNA extraction (DECAMP-1, Lahey).

Thawed nasal brush specimens in RNAprotect were agitated to remove cells from the brush either by vortexing or using a Tissuelyser without bead (QIAGEN, Hilden, Germany) and then cells were pelleted by centrifugation (5000-10000 g, 5 min). Following removal of RNAprotect, the cell pellet was lysed using Qiazol reagent and total RNA extracted using the miRNeasy Mini Kit (QIAGEN, Hilden, Germany) according to the manufacturer's instructions. RNA quantification was performed using the QuantiFluor RNA System (Promega, Madison, WI), and 50 ng of RNA was used as input to the TruSeq RNA Access Library Prep procedure (Illumina, San Diego, CA), which enriches for the coding transcriptome. Libraries meeting quality control criteria for amplification yields were sequenced using NextSeq 500/550 instruments (2×75 bp paired-end reads) with the High Output Kit (Illumina, San Diego, CA).

Raw sequencing (FASTQ) files were aligned to the Human Reference assembly 37 (Genome Reference Consortium) using the STAR RNA-seq aligner software. Uniquely mapped and non-duplicate reads were summarized for 63,677 annotated Ensembl genes using HTSeq. Data quality metrics were generated using RNA-SeQC. Samples were excluded and re-sequenced when their library sequence data did not achieve minimum criteria for total reads, uniquely mapped reads, mean per-base coverage, base duplication rate, percentage of bases aligned to coding regions, base mismatch rate and uniformity of coverage within each gene. To monitor and evaluate technical batch effects, nasal brushing samples from five patients (sentinels) were included in each 96-well plate across all sequencing runs. Kinship analysis was performed on all samples with acceptable sequencing quality metrics to ensure sample identity.

### Normalization and Gene Filtering

Sequence data were filtered to exclude features not targeted for enrichment by the assay, resulting in a total feature set of 26,268 Ensembl genes. Expression count data were normalized by the variance stabilizing transformation (VST) method in DESeq2. Principal component analysis (PCA) was performed in sentinels or patient samples to evaluate overall variability.

909 genes were identified and excluded with high technical variabilities among sentinels. Genes were also excluded when the 75th percentiles of expression values were less than 6 among patient samples. After these exclusions, 14,897 gene features were eligible for downstream analysis. Top principal components from PCA were regressed out of expression values to control for large variabilities which may confound downstream analysis.

### Genomic Indexes

Novel genomic indexes were developed for sex, smoking status, and smoking burden. Given that blood contamination could impact classifier performance, Hemoglobin Subunit Beta gene expression was used to measure the degree of contamination and used as a prospective exclusion criterion

### Classifier Development

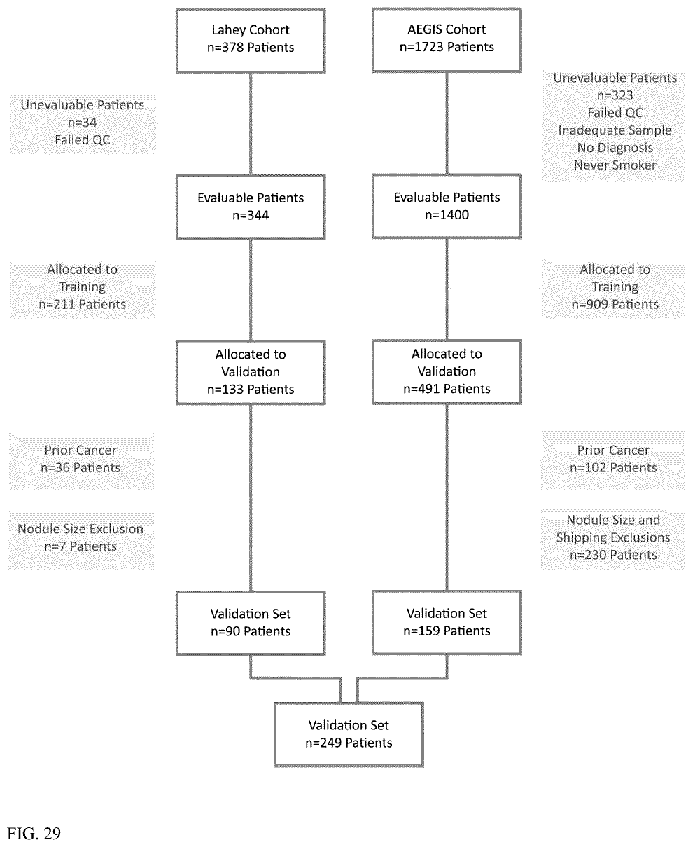

The classifier was designed to yield low, intermediate and high categories to conform to current PN management guidelines. Candidate classifiers were developed using samples allocated to training (FIG. 29). Parameter optimization, performance evaluation and model selection were conducted using cross-validation within the training set. Hyper-parameter tuning was used to determine values for the final classifier. The classifier can be hierarchical in structure consisting of an up-stream and a down-stream model. The former can be a penalized logistic regression model with age, nodule length, nodule spiculation, years since quit, and genomic smoking duration index as covariates, focused on identifying PN as high-risk. The remaining patients were evaluated by the down-stream model and further stratified to low/intermediate/high-risk. The down-stream model can be a Support Vector Machine incorporating interaction terms between gene and clinical covariates, including age, nodule length, nodule spiculation, and pack-years, as well as interactions between genes and the genomic indexes. The classifier can comprise genes as provided in Table 1, including ones used in the classifier and in the genomic indexes. The classifier genes and genomic indexes were assessed for biological function and involvement in known signaling pathways using Enrichr analysis.

The classifier can have a hierarchical structure and can consist of an up-stream model and a down-stream model. The up-stream model can be a penalized logistic regression model with age, nodule length (log2 transformed), nodule spiculation (Y/N), years since quit and genomic smoking duration index as covariates. When the patient's prediction value is higher than 0.8932, the patient can be classified as high-risk, otherwise, the patient can be evaluated by the down-stream model. The down-stream model can be a Support Vector Machine incorporating the following features: age, nodule length (log2 transformed), nodule spiculation (Y/N), pack-year, genomic sex, genomic smoking duration index, genomic smoking status (current vs. former) index as well as genes selected using Differential Expression analysis. In the down-stream model, when the patient's prediction value is higher than 0.8768, the patient can be classified as high-risk. When the patient's prediction value is lower than −1.4348, the patient can be classified as low-risk. The remaining patients between these values can be classified as intermediate risk.

### Example 18: Statistical Analysis

The 95% confidence intervals for sensitivity, specificity, NPV and PPV were calculated using Wilson's method. A one-sided z-test with continuity correction was used for a comparison of the classifier to three validated clinical risk models: the Veteran's Affairs (VA) Model, Mayo Model, and Brock1b Model.

When calculating sensitivity, specificity and PPV for high-risk classification, high-risk calls are counted as positive calls and intermediate and low-risk calls are counted as negative (not-high-risk) calls. When calculating sensitivity, specificity and NPV for low-risk classification, high and intermediate-risk calls were counted as positive calls (not-low-risk) and low-risk calls were counted as negative calls. Classifier performance was compared to three validated clinical risk models: the VA Model1, Mayo Model2, and Brock1b Model3, confining the analysis to nodules 8-30 mm to conform to the size range included in the validation cohorts of the models.

Sensitivity for low-risk classification is 96% with specificity of 42%. Specificity of high-risk classification is 90% with sensitivity of 58%. Extrapolated to a prevalence of 25%, the negative predictive value for low-risk classification is 97%, and the positive predictive value for high-risk classification is 67%. No malignant PN ≥8 mm were labeled low-risk. Two thirds of malignant PN<8 mm were labeled intermediate-risk. Sensitivity was similar across stages of non-small cell lung cancer, independent of subtype. Performance compared favorably to clinical-only risk models. Analysis of 63 patients with prior cancer shows similar performance.

The nasal classifier provides accurate assessment of ROM in individuals who smoke with a PN. Classifier-guided decision-making could lead to fewer unnecessary diagnostic procedures in patients without cancer and more timely treatment in patients with lung cancer.

### Example 19—Independent Classifier Validation

The final classifier was evaluated for the primary endpoint on an independent, prospectively defined validation set of 249 patients. NPV of the low-risk classification and PPV of the high-risk classification were calculated on the 249-patient validation set at the study prevalence of malignancy, and then extrapolated to 25% cancer prevalence to better match the expected clinical use population of the classifier. Subgroup analyses were conducted for nodule size, cancer stage, and histologic subtype. The protocol specified that once the primary endpoint was achieved, an additional 63 patients with prior cancer other than lung cancer would be evaluated. These patients met all other inclusion and exclusion criteria, including exclusion for prior lung cancer.

### Example 20—Performance of the Clinical-Genomic Classifier in the Primary Validation Set

In the combined primary validation set and the prior cancer set, the classifier demonstrated 98% NPV and 70% PPV for low-risk and high-risk classification, respectively, in a population with 25% cancer prevalence.

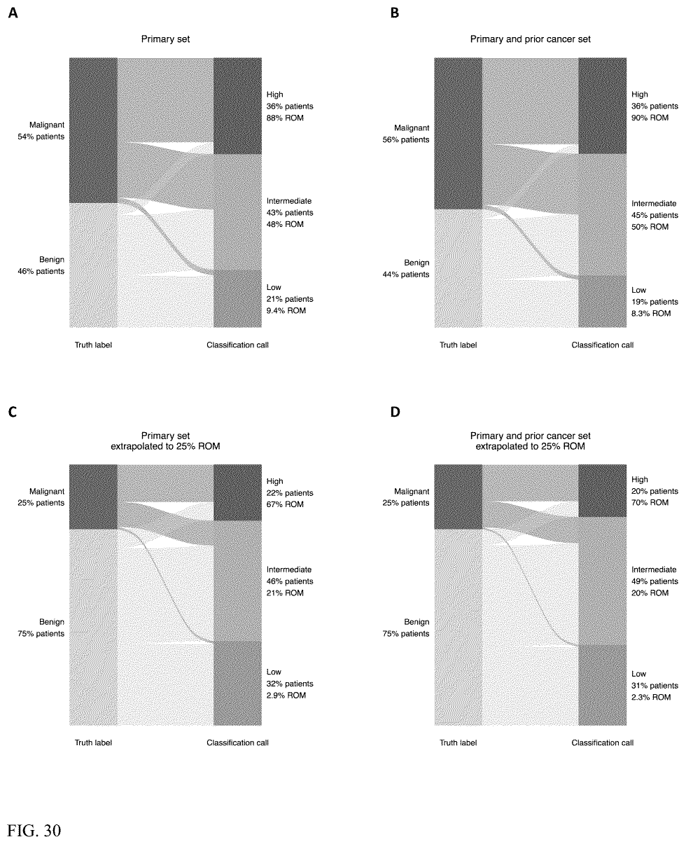

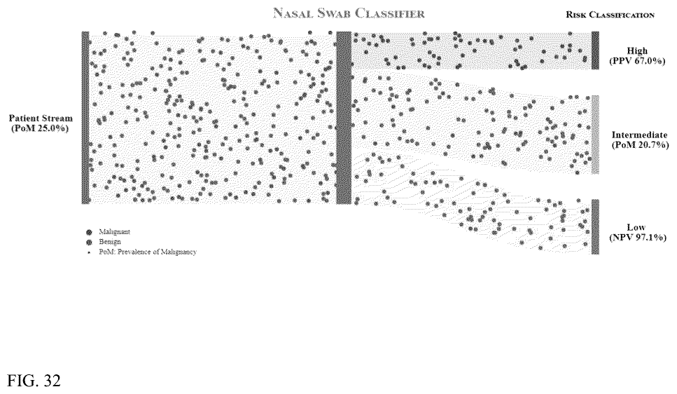

Demographics and nodule characteristics for the 249 patients in the primary validation set are shown in Table 43. Table 41 shows the distribution of PN in the three risk classifications. In the group of 115 benign nodules, 48 (42%) were classified as low, 56 (49%) as intermediate, and 11 (10%) as high-risk. In the group of 134 malignant nodules, 5 (4%) were classified as low, 51 (38%) as intermediate, and 78 (58%) as high-risk. A Sankey plot showing relative distribution of the primary validation set into low, intermediate and high-risk categories in a population extrapolated to 25% cancer prevalence is shown in FIG. 32. Alluvial diagrams showing the distribution of benign and malignant nodules into three risk categories are shown in FIG. 30.

TABLE 41
Performance of the nasal genomic classifier in the primary validation
set, showing classifier results for benign and malignant nodules.

Primary Validation Set

| Nasal Swab Risk Stratification | Benign | Malignant |
|---|---|---|
| # High-Risk | 11 (10%) | 78 (58%) |
| # Intermediate-Risk | 56 (49%) | 51 (38%) |
| # Low-Risk | 48 (42%) | 5 (4%) |
| Total | 115 | 134 |

TABLE 42
Classifier performance (sensitivity, specificity, and
PPV or NPV at a cancer prevalence of 25%) for the high-
risk classification and the low-risk classification.

| | Primary Validation Set | | |
|---|---|---|---|
| Nasal Swab Risk | | | Extrapolated |
| Stratification | Sensitivity | Specificity | to 25% ROM |
| High-Risk vs. not High-Risk | 58 | 90 | PPV |
| (Intermediate + Low) | (50-66) | (84-95) | 67 (54-78) |
| Low-Risk vs. not Low-Risk | 96 | 42 | NPV |
| (Intermediate + High) | (92-98) | (33-51) | 97 (91-99) |

(95% CI in parenthesis)

TABLE 43
Demographics and nodule characteristics for the patients
included in the primary validation set (n = 249)

| | | PRIMARY SET | |
|---|---|---|---|
| | | Benign | Malignant |
| Category | Sub-category | n = 115 | n = 134 |
| Age* | Median | 63 | 66 |
| Sex \| n (%) | M | 66 (57.4%) | 85 (63.4%) |
| | F | 49 (42.6%) | 49 (36.6%) |
| Race \| n (%) | White | 106 (92.2%) | 115 (85.8%) |
| | Black/African | 6 (5.2%) | 16 (11.9%) |
| | American | | |
| | Other | 2 (1.7%) | 3 (2.2%) |
| | Unknown | 1 (0.9%) | 0 (0%) |
| Smoking \| n (%) | Current | 46 (40.0%) | 65 (48.5%) |
| | Former | 69 (60.0%) | 69 (51.5%) |
| Pack-Years* | Median | 36 | 50 |
| Years since quit* | Median | 11 | 6 |
| (in former smokers) | | | |
| COPD \| n (%) | Yes | 34 (29.6%) | 66 (49.3%) |
| | No | 80 (69.6%) | 67 (50.0%) |
| | Unknown | 1 (0.9%) | 1 (0.7%) |
| Nodule Size* | <1 | 71 (61.7%) | 20 (14.9%) |
| (cm) \| n (%) | 1-2 | 31 (27.0%) | 56 (41.8%) |
| | >2-3 | 13 (11.3%) | 58 (43.3%) |
| Spiculation* \| n (%) | Yes | 9 (7.8%) | 40 (29.9%) |
| | No | 106 (92.2%) | 94 (70.1%) |
| Nodule | Upper lobe | 34 (29.5%) | 75 (56.0%) |
| location \| | Non-upper lobe | 63 (54.8%) | 48 (35.8%) |
| n (%) | Unknown | 18 (15.7%) | 11 (8.2%) |
| Histology \| n (%) | NSCLC | | 102 (76.1%) |
| | SCLC | | 19 (14.2%) |
| | Other/Unknown | | 13 (9.7%) |
| NSCLC type \| n (%) | Adenocarcinoma | | 51 (50.0%) |
| | Squamous Cell | | 36 (35.3%) |
| | Large Cell | | 2 (2.0%) |
| | Other/Unknown | | 13 (12.7%) |

*Clinical features included in the 502 gene clinical-genomic classifier.

Sensitivity and Specificity for each decision boundary are shown in Table 42. Sensitivity for the low-risk classification was 96% (95% CI 92%-98%) at a specificity of 42% (95% CI 33%-51%). The high-risk classification specificity was 90% (95% CI 84%-95%) with a sensitivity of 58% (95% CI 50%-66%). At the study prevalence of 54% malignancy, NPV is 91% for the low-risk classification and PPV is 88% for the high-risk classification. With data extrapolated to a 25% cancer prevalence, NPV for low-risk classification is 97%, and PPV for high-risk classification is 67% (Table 42).

### Classifier Performance by Nodule Size

Performance of the classifier was evaluated in PN<8 mm and 8-30 mm. The classifier labeled ⅔ of malignant nodules ≥8 mm in size as high-risk (66%) and the remainder as intermediate-risk (34%) (Table 30), demonstrating a 100% (95% CI 97%-100%) sensitivity for low vs. not-low-risk classification (Table 30 and Table 31). The classifier labeled ⅔ of all malignant nodules<8 mm as intermediate-risk, retaining a 67% (95% CI 42%-85%) sensitivity for low vs. not-low-risk classification. The classifier labeled all benign PN<8 mm in size as low (63%) or intermediate (37%) risk, demonstrating a 100% (95% CI 84%-100%) specificity for high vs. not-high-risk classification. For benign PN ≥8 mm, the majority were classified as low (15%) or intermediate (63%) risk, retaining a 78% (95% CI 66%-88%) specificity.

TABLE 30
Classifier results in the primary validation set
comparing PN < 8 mm vs. ≤ 8 mm.

| Nodule Length | Nodule < 8 mm | | Nodule ≥ 8 mm | |
|---|---|---|---|---|
| Patient label | Benign | Malignant | Benign | Malignant |
| # High-Risk | 0 (0%) | 0 (0%) | 11 (21%) | 78 (66%) |
| # Intermediate-Risk | 23 (37%) | 10 (67%) | 33 (63%) | 41 (34%) |
| # Low-Risk | 40 (63%) | 5 (33%) | 8 (15%) | 0 (0%) |
| Total | 63 | 15 | 52 | 119 |

TABLE 31
Classifier performance (sensitivity and specificity) for the high-risk classification
and the low-risk classification comparing PN < 8 mm vs. ≤ 8 mm.

| Nasal Swab Risk | Nodule < 8 mm | | Nodule ≥ 8 mm | |
|---|---|---|---|---|
| Stratification | Sensitivity | Specificity | Sensitivity | Specificity |
| High-Risk vs. not High-Risk | 0 (0-20) | 100 (94-100) | 65.55 (57-73) | 78.85 (66-88) |
| (Intermediate + Low) | | | | |
| Low-Risk vs. not Low-Risk | 66.67 (42-85) | 63.49 (51-74) | 100 (97-100) | 15.38 (8-28) |
| (Intermediate + High) | | | | |

Performance with VA, M and B1b Models

Comparison of low-risk classification fixed at the same sensitivity shows that the classifier's specificity is significantly better than the VA model (p=0.019) and shows moderate improvement to B1b (p=0.06) (Table 32 and Table 33). For high-risk classification fixed at the same specificity, the classifier's sensitivity is significantly better than M(p=0.037) and B1b (p=0.003). The classifier labeled significantly more benign patients as low-risk compared to the VA Model. The classifier labeled significantly more patients with lung cancer as high-risk compared to M and B1b.

TABLE 32
Comparison of the nasal genomic classifier to clinical
risk models. For the low-risk classification, the models
were fixed at the same sensitivity, and for the high-risk
classification, the models were fixed at the same specificity.
Comparison to the VA (Veteran's Affairs) Model

| Nasal Swab Risk | | | | |
|---|---|---|---|---|
| Stratification | Classifier | Sensitivity | Specificity | p-value |
| High-risk | Nasal Classifier | 58.21 | 90.43 | 0.5 |
| | VA Model | 57.46 | | |
| Low-risk | Nasal Classifier | 96.27 | 41.74 | 0.019* |
| | VA Model | | 27.83 | |

TABLE 33
Comparison of the nasal genomic classifier to clinical risk models.
For the low-risk classification, the models were fixed at the same
sensitivity, and for the high-risk classification, the models were
fixed at the same specificity. Comparison the M and B1b Models.

| Nasal Swab Risk | | | | |
|---|---|---|---|---|
| Stratification | Classifier | Sensitivity | Specificity | p-value |
| High-Risk | Nasal Classifier | 59.35 | 89.69 | |
| | M | 47.15 | | 0.037* |
| | B1b | 40.65 | | 0.003* |
| Low-Risk | Nasal Classifier | | 36.08 | |
| | M | 98.37 | 39.18 | 0.62 |
| | B1b | | 24.74 | 0.06 |

* p-value<0.05 for comparison of Specificity

### Classifier Performance by Cancer Stage and Histologic Subtype in Malignant Nodules

Performance of the classifier is similar across all four stages of NSCLC (Table 39 and Table 40), with good sensitivity for the high-risk classification across all stages of NSCLC and limited stage Small Cell Lung Cancer (SCLC). The classifier labeled no patient with NSCLC Stage II or greater as low-risk, retaining a 100% sensitivity for low-risk classification. Histology was available for 121 (90%) of the 134 patients with lung cancer (Table 34). In 102 NSCLC patients, the classifier categorized 57% patients with adenocarcinoma and 72% patients with squamous cell carcinoma to high-risk while maintaining 97% NSCLC patients in the intermediate or high-risk categories. (Table 35).

TABLE 39
Classifier results and by stage in patients in the primary
data set ultimately diagnosed with lung cancer (n = 134).

Nasal Swab

| Risk | Cancer Stage | | | | | | | | | | | | | |
|---|---|---|---|---|---|---|---|---|---|---|---|---|---|---|
| Stratification | Stage 1* | | Stage 2* | | Stage 3* | | Stage 4* | | Extensive^{†} | | Limited^{†} | | Missing | |
| # High-Risk | 26 | (55%) | 3 | (60%) | 12 | (67%) | 14 | (58%) | 4 | (44%) | 5 | (56%) | 14 | (64%) |
| # Intermediate- | 18 | (38%) | 2 | (40%) | 6 | (33%) | 10 | (42%) | 3 | (33%) | 4 | (44%) | 8 | (36%) |
| Risk | | | | | | | | | | | | | | |
| # Low-Risk | 3 | (6%) | 0 | (0%) | 0 | (0%) | 0 | (0%) | 2 | (22%) | 0 | (0%) | 0 | (0%) |

| | | | | | | | |
|---|---|---|---|---|---|---|---|
| Total | 47 | 5 | 18 | 24 | 9 | 9 | 22 |

TABLE 40
Classifier performance (shown as sensitivity for the high-risk and low-risk classifications) and
by stage in patients in the primary data set ultimately diagnosed with lung cancer (n = 134).

Nasal Swab

| Classification | Cancer Stage | | | | | | |
|---|---|---|---|---|---|---|---|
| Sensitivity | Stage 1* | Stage 2* | Stage 3* | Stage 4* | Extensive^{†} | Limited^{†} | Missing |
| High-Risk vs. | 55 | 60 | 67 | 58 | 44 | 56 | 64 |
| not High-Risk | (41-69) | (23-88) | (44-84) | (39-76) | (19-73) | (27-81) | (43-80) |
| (Intermediate + | | | | | | | |
| Low) | | | | | | | |
| Low-Risk vs. | 94 | 100 | 100 | 100 | 78 | 100 | 100 |
| not Low-Risk | (83-98) | (57-100) | (82-100) | (86-100) | (45-94) | (70-100) | (85-100) |
| (Intermediate + | | | | | | | |
| High) | | | | | | | |

Sensitivity (95% CI in parenthesis)
*Non-Small Cell Lung Cancer
†Small Cell Lung Cancer

TABLE 34
Classifier results in the primary validation, Non-
Small Cell Lung Cancer (NSCLC), Small Cell Lung
Cancer (SCLC), and histology unknown (missing).

| Nasal Swab Risk | Cell Type | | |
|---|---|---|---|
| Stratification | Missing | NSCLC | SCLC |
| # High-Risk | 6 (46%) | 63 (62%) | 9 (47%) |
| # Intermediate-Risk | 7 (54%) | 36 (35%) | 8 (42%) |
| # Low-Risk | 0 (0%) | 3 (3%) | 2 (11%) |
| Total | 13 | 102 | 19 |

TABLE 35
Classifier results in the primary validation
set for NSCLC histologic subtypes.

| | |
|---|---|
| Nasal Swab Risk | NSCLC Histology |

| Stratification | Adenocarcinoma | Other | Squamous |
|---|---|---|---|
| # High-Risk | 29 (57%) | 8 (53%) | 26 (72%) |
| # Intermediate-Risk | 20 (39%) | 6 (40%) | 10 (28%) |
| # Low-Risk | 2 (4%) | 1 (7%) | 0 (0%) |
| Total | 51 | 15 | 36 |

Patients with a History of Prior Cancer

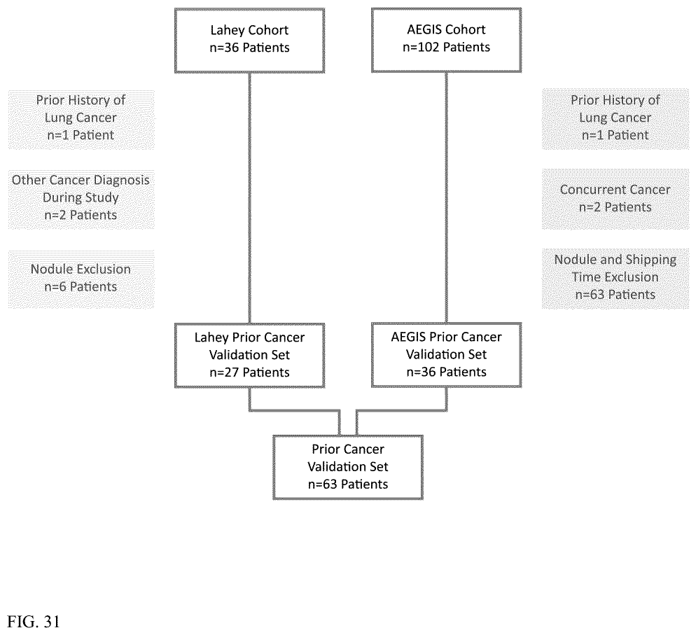

The prior cancer set consisted of 63 patients, of whom approximately half had a prior solid organ or hematologic malignancy, and half had a non-melanoma skin cancer (FIG. 31 and Table 36). In this group the classifier labeled no patients with a malignant PN as low-risk and labeled no patients with a benign PN as high-risk (Table 37), resulting in a 100% specificity for the high-risk classification and 100% sensitivity for the low-risk classification. With the two sets combined (n=312), the NPV and PPV in a population with a 25% cancer prevalence are 98% and 70% for the low-risk and high-risk classification, respectively (Table 38). ROM in the intermediate-risk group is 2% (95% CI 14.8-27.6).

TABLE 36
Patients in the set with a prior cancer (excluding lung
cancer) for the AEGIS cohorts and Lahey cohort.

| Cancer type | AEGIS | Lahey |
|---|---|---|
| basal cell | 7 | 12 |
| bladder | 5 | 2 |
| breast | 3 | 5 |
| cervical | 2 | 0 |
| colon | 3 | 1 |
| esophageal | 1 | 0 |
| head neck | 5 | 0 |
| leukemia | 1 | 0 |
| liver | 1 | 0 |
| lymphoma | 1 | 1 |
| melanoma | 1 | 2 |
| prostate | 5 | 2 |
| rectal | 0 | 1 |
| renal | 1 | 1 |
| skin unknown | 5 | 0 |
| squamous cell | 2 | 5 |
| uterine | 1 | 0 |

TABLE 37
Classifier results in the prior cancer set and the prior
cancer set combined with the primary validation set.

| Nasal Swab Risk | Prior Cancer Set (n = 63) | | Combined (n = 312) | |
|---|---|---|---|---|
| Stratification | Benign | Malignant | Benign | Malignant |
| # High-Risk | 0 (0%) | 22 (54%) | 11 (8%) | 100 (57%) |
| # Intermediate-Risk | 15 (68%) | 19 (46%) | 71 (52%) | 70 (40%) |
| # Low-Risk | 7 (32%) | 0 (0%) | 55 (40%) | 5 (3%) |
| Total | 22 | 41 | 137 | 175 |

TABLE 38
Classifier performance (sensitivity, specificity, and PPV or NPV at a cancer prevalence
of 25%) for the high-risk classification and the low-risk classification.

| Nasal Swab | Prior Cancer | | | Combined | | |
|---|---|---|---|---|---|---|
| Risk | | | Extrapolated | | | Extrapolated |
| Stratification | Sensitivity | Specificity | to 25% ROM | Sensitivity | Specificity | to 25% ROM |
| High-Risk vs. | 54 | 100 | PPV | 57 | 92 | PPV |
| not High-Risk | (39-68) | (85-100) | 100 (69-100) | (50-64) | (86-95) | 70 (58-80) |
| (Intermediate + | | | | | | |
| Low) | | | | | | |
| Low-Risk vs. | 100 | 32 | NPV | 97 | 40 | NPV |
| not Low-Risk | (91-100) | (16-53) | 100 (80-100) | (93-99) | (32-49) | 98 (92-99) |
| (Intermediate + | | | | | | |
| High) | | | | | | |

### Example 21—Pathway Analysis of the 502 Gene Classifier

The genes within the nasal classifier and genomic smoking indexes were assessed for biological function and involvement in known signaling pathways using the Enrichr functional annotation tool. The nasal classifier genes work in partnership with clinical variables, and it is therefore not as straightforward to interpret their function through pathway investigation. As expected, though containing many genes with known cell signaling function, the nasal classifier gene set was not found to be highly enriched for canonical signaling pathways. However, analysis of the smoking genomic indexes did identify conceptually plausible pathways enriched for index genes. This includes the nicotine degradation pathway containing index genes cytochrome p450 CYP4X1 and AOX1 whose expression in the airway has been shown to be regulated by cigarette smoke exposure. Additionally, pathways involved in cadherin and WNT signaling, extracellular matrix organization and epithelial mesenchymal transition were identified, all of which have previously been associated with the response to cigarette smoke.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-57. (canceled)

58. A method for determining that a subject is not at an elevated risk of having lung cancer, comprising (a) assaying a biological sample from a nasal passageway of said subject for a level of expression, and (b) processing said level of expression to determine that said subject is not at said elevated risk of having said lung cancer at a specificity of at least 51%.

59. The method of claim 58, wherein (b) is performed at a sensitivity of at least 95%.

60. The method of claim 58, wherein said biological sample is a sample of airway epithelial cells.

61. The method of claim 60, wherein said airway epithelial cells are obtained by a nasal swab.

62. The method of claim 58, wherein said lung cancer comprises one or more of non-small cell lung cancer, a small cell lung cancer, a lung carcinoid tumor, or a bronchial carcinoid tumor.

63. The method of claim 62, wherein said non-small cell lung cancer comprises one or more of an adenocarcinoma, a squamous cell carcinoma, or a large cell carcinoma.

64. The method of claim 63, wherein processing comprises correlating one or more additional levels of expression with one or more genomic index.

65. The method of claim 64, wherein said one or more genomic index comprises a blood contamination index, a smoking duration index, a smoking status index, a cell type normalization index, a genomic gender index, or a combination thereof.

66. The method of claim 65, wherein said blood contamination index comprises an expression level of Hemoglobin Subunit Beta.

67. The method of claim 65, wherein said smoking duration index comprises an expression level of one or more genes selected from Table 1.

68. The method of claim 65, wherein said smoking duration index comprises an expression level of one or more genes selected from the group consisting of: AC074091.1, ACTL10, ADRA2B, AGT, ALDOC, AMACR, AOX1, APEH, APOPT1, ARHGEF35, ARNTL, ATF7IP2, ATP2A3, BBOX1, BHLHE40-AS1, BNIP3, BOLA1, BPI, C11orf68, C12orf65, C1QL2, C21orf128, C2orf73, CACNA1B, CAPG, CAPN9, CDC25A, CDC42P6, CDCA2, CDCP1, CDHR1, CDHR2, CDK5, CDNF, CMTM2, COG1, COL1A1, COL5A3, CORO2B, CST7, CTD-2555O16.2, CTD-2555O16.4, CTGLF12P, CTNS, CTSF, CXCL12, CYP7B1, DBI, DDO, DDT, DLL1, DOCK3, DRD4, EDIL3, EFHB, ETFDH, EVA1A, FAM184A, FAM189B, FLT1, FOXC2, FTCDNL1, GALNT16, GET4, GLB1L3, GNAL, GNG4, GOLGA8O, GOT1, HARBI1, HAUS4, HCAR3, HERC2P2, HIST1H3E, HIST1H4F, HLA-J, HORMAD1, HSF4, HSF5, IGF2BP2, ISYNA1, KCNMB3, KCNQ3, KCTD10, KDR, KIAA0513, KRT39, KRT40, KRTAP5-7, LOXHD1, LTBP1, LUZP2, LYRM5, MAD2L1BP, MMD, MMP1, MPP7, MRM1, MRPS6, MRVI1-AS1, MUC6, MUT, MVB12A, NAMPTL, NBR2, NDUFA6, NDUFAF6, NDUFS7, NEFH, NLRP2, NME6, NPSR1, NUDT7, OLFM1, ORAOV1, PALM3, PAPSS1, PCDHA12, PCDHA13, PCDHB11, PCDHB16, PDPR, PEX11A, PIAS2, PIPOX, PLAG1, PLG, PMP22, PMS2P5, POLR2M, PPFIA3, PPP1R42, PRPF38B, PTGER4, RANGRF, RBMS3, RIMBP2, RIMKLA, RND2, RP11-163E9.2, RP11-171I2.2, RP11-171I2.4, RP11-345J4.8, RP11-461A8.1, RP11-477D19.2, RP11-522I20.3, RP11-695J4.2, RPL9, RUSC1, SCN11A, SDHAF2, SEMA3F, SEPT7P9, SFRP2, SH3GL3, SLAMF6, SLC22A3, SLC37A2, SLC48A1, SLC6A13, SNORD101, SP6, SPINK1, STAG3L2, STXBP5L, TEKT4, TERF2, TF, TFAP2C, TMEM200C, TMEM213, TMTC4, TP53I11, TTC39B, TTLL13, TWF2, TYRO3, UBAP1L, WDR53, WIPF3, ZFP2, ZFP28, ZNF232, ZNF576, and ZNF624.

69. The method of claim 65, wherein said smoking status index comprises an expression level of one or more genes selected from Table 1.

70. The method of claim 65, wherein said smoking status index comprises an expression level of one or more genes selected from the group consisting of: ACVRL1, AHRR, AP1S3, ARRDC4, B3GNT6, BAALC, BPIFB2, CACNA2D3, CCDC69, CCDC88A, CD163L1, CDK5RAP2, CIT, CLIC5, CMTM7, CNGB1, COL1A2, COL3A1, COL6A3, CPE, CPNE8, CRNN, CYP2A13, CYP4X1, EDC3, ENC1, ENTPD8, FHL1, FOXE1, GAD1, GLDN, GLYATL2, GRAMD2, GSTO2, hsa-mir-7162, HSF4, ICA1, IGF1, IL36A, JAKMIP3, KPRP, LCE3D, LRRC31, MAMDC2, MGP, MMP7, MPST, NOL3, NOX4, NRIP1, OCA2, PANX2, PBX3, PRKAR2B, RAMP1, RDH10, RHCG, RNF175, RPTN, SAA1, SAA2, SAMHD1, SERPINE2, SETD7, SLC16A12, SLC28A2, SLPI, TGM3, TGM6, TIPARP, TMEM45B, TRHDE, TRNAU1AP, UCHL1, USH1C, USP54, WNTSA, and ZKSCAN1.

71. The method of claim 65, wherein processing comprises regressing out said one or more additional levels of expression associated with said cell type normalization index.

72. The method of claim 65, wherein said genomic gender index comprises an expression level of one or more of USP9Y, RPS4Y1, UTY, DDX3Y, or KDM5D.

73. The method of claim 58, further comprising measuring one or more additional levels of expression to determine an integrity of ribonucleic acid (RNA) in said sample.

74. The method of claim 58, further comprising measuring one or more clinical covariates comprising one or more of age, nodule length, nodule spiculation, or pack years.

75. The method of claim 58, wherein processing comprises applying a trained classifier.

76. The method of claim 75, wherein said trained classifier is trained using gene expression data from subjects diagnosed with lung cancer.

77. The method of claim 76, wherein said subjects diagnosed with lung cancer include subjects with lung nodule sizes between 6 mm and 30 mm in diameter, subjects with lung nodule sizes less than 6 mm in diameter, subjects with unknown lung nodules size, or a combination thereof.

78. A method for determining that a subject is not at an elevated risk of having lung cancer, comprising (a) assaying a biological sample from a nasal passageway of said subject for a level of expression, and (b) processing said level of expression to determine that said subject is not at said elevated risk of having said lung cancer at a sensitivity of at least 60%.
